SEQUENCE LISTING (6 sequences)

SEQ ID NO: 1 - DNA, 52 bp, Mus musculus
taccctggccttcctggtactcagcacacctgctatgtttgacagagccctc

SEQ ID NO: 2 - protein, 18 aa, Mus musculus
TLAFLVLSTPAMFDRALK

SEQ ID NO: 3 - DNA, 50 bp, Artificial Sequence
  portion of exon 2 of del3
taccctggccttcctggtactcagacctgctatgtttgacagagccctca

SEQ ID NO: 4 - protein, 17 aa, Artificial Sequence
  protein encoded by portion of exon 2 of del3
TLAFLVLRPAMFDRALK

SEQ ID NO: 5 - DNA, 51 bp, Artificial Sequence
  portion of exon 2 of del2
taccctggccttcctggtactcagcacctgctatgtttgacagagccctca

SEQ ID NO: 6 - protein, 12 aa, Artificial Sequence
  protein encoded by portion of exon 2 of del2
TLAFLVLSTCYV

(19) United States
(12) Patent Application Publication
(10) Pub. No.: US 2023/0390322 A1
(43) Pub. Date: Dec. 7, 2023

(54) HIGH CONCENTRATION METHYLCOBALAMIN OR COMBINATION OF METHYL- AND HYDROXY-COBALAMIN FOR THE TREATMENT OF COBALAMIN C DEFICIENCY DISORDERS

(71) Applicant: THE U.S.A., as represented by the Secretary, Department of Health and Human Services, Bethesda, MD (US)

(72) Inventors: Charles P. Venditti, Potomac, MD (US); Jennifer Lyn Sloan, Bethesda, MD (US); Eirini Manoli, Rockville, MD (US)

(73) Assignee: THE U.S.A., as represented by the Secretary, Department of Health and Human Services, Bethesda, MD (US)

(21) Appl. No.: 18/032,132

(22) PCT Filed: Oct. 12, 2021
(86) PCT No.: PCT/US2021/054619

§ 371 Date: Apr. 14, 2023

Related U.S. Application Data
(60) Provisional application No. 63/093,084, filed on Oct. 16, 2020.

(51) Int. Cl. A61K 31/714; A61P 3/02

(52) U.S. Cl. CPC A61K 31/714; A61P 3/02

(57) ABSTRACT

Methods are disclosed for treating a subject with a cobalamin C (cblC) deficiency. These methods include selecting a human subject with the cblC deficiency; and administering about 5 mg to about 10 grams of methylcobalamin (MeCbl) daily to the human subject. Methods also are disclosed for treating a fetus with a cobalamin C (cblC) deficiency. The methods include selecting a female human subject pregnant with the fetus that has the cblC deficiency; and administering about 5 mg to about 10 grams of MeCbl daily to the female human subject, in order to treat the cblC deficiency in the fetus. Optionally, OHCbl can be administered to the subject.

### CROSS REFERENCE TO RELATED APPLICATIONS

This claims priority to U.S. Application No. 63/093,084, filed Oct. 16, 2020, which is incorporated herein by reference.

### STATEMENT OF GOVERNMENT SUPPORT

This invention was made with Government support under project number Z01 #: 1ZIAHG200318-15 by the National Institutes of Health, National Human Genome Research Institution. The United States Government has certain rights in the invention.

### FIELD OF THE DISCLOSURE

This relates to the treatment of disorders of a cobalamin C (cblC) deficiency, with the use of a high dose of methylcobalamin (MeCbl).

### BACKGROUND

Vitamin B12 is an essential component of the mammalian diet and its deficiency causes significant morbidity worldwide (Stabler, N Engl J Med 368, 2041-2 (2013)). B₁₂ deficiency can be identified by biochemical manifestations of methylmalonic acidemia and hyperhomocysteinemia, due to the deficiency of the intracellular cofactors 5′-deoxyadenosylcobalamin (AdoCbl) and MeCbl required for the enzymatic reactions methylmalonyl-CoA mutase and methionine synthase, respectively.

There are many genes responsible for the processing and intracellular trafficking of vitamin B12. In particular, MMACHC plays a critical role in the transport and synthesis of these cofactors by removing the upper axial ligand of cob(III)alamin derivatives obtained from the diet and supplementation (e.g. hydroxocobalamin (OHCbl) or cyanocobalamin (CNCbl)) resulting in a cob(II)alamin intermediate from which AdoCbl and MeCbl are derived (Mascarenhas, et al., J Biol Chem 295, 9630-9640 (2020); Hannibal et al., Mol Genet Metab 97, 260-6 (2009)).

Pathogenic variants in MMACHC cause the most common inborn error of intracellular cobalamin metabolism, combined methylmalonic acidemia with hyperhomocysteinemia cblC type (cblC) (Lerner-Ellis et al., Hum Mutat 30, 1072-81 (2009)) affecting approximately 1 in 60-100,000 births in the US (Weisfeld-Adams et al., Mol Genet Metab 99, 116-23 (2010)), with a reported incidence 1 in 4000 in some regions in China (Han et al., Brain Dev 38, 491-7 (2016). Historically the clinical presentations of cblC range throughout the lifespan from in utero to adulthood (Sloan et al., “Disorders of Intracellular Cobalamin Metabolism,” in GeneReviews((R)) (eds. Adam, M. P. et al.) (Seattle (WA), 1993); Carrillo-Carrasco and Venditti, J Inherit Metab Dis 35, 103-14 (2012)) and are clinically heterogeneous demonstrating primarily neurologic, hematologic and ophthalmologic manifestations. There is high mortality in cblC if the disorder is not promptly recognized and treated with high doses of injectable OHCbl (Rosenblatt et al., J Inherit Metab Dis 20, 528-38 (1997); Fischer et al., J Inherit Metab Dis 37, 831-40 (2014)). While medical management with OHCbl, betaine, folinic acid and optimal protein intake can improve survival, biochemical parameters and some clinical symptoms (Carmel et al., Blood 55, 570-9 (1980); Mamlok et al., Neuropediatrics 17, 94-9 (1986); Bartholomew, et al., J Pediatr 112, 32-9 (1988); Huemer et al., J Inherit Metab Dis 40, 21-48 (2017)), many patients still develop neurological complications such as seizures, hydrocephalus (He R, et al., Neurology. doi: 10.1212/WNL.0000000000010912. Epub ahead of print. PMID: 32943488 (2020 Sep. 17)), intellectual disability and an ocular syndrome characterized by a “bulls-eye” maculopathy with a progressive retinal degeneration and blindness (Brooks et al., Ophthalmology 123(3):571-82 (2016); (He R, et al., Neurology. doi: 10.1212/WNL.0000000000010912. Epub ahead of print. PMID: 32943488 (2020 Sep. 17). In the USA and many other countries, cblC deficiency is detected through expanded newborn screening (Huemer et al., J Inherit Metab Dis 38, 1007-19 (2015); Weisfeld-Adams et al., Mol Genet Metab 110, 241-7 (2013)), yet treatments for cblC deficiency lack evidenced based guidelines and the underlying disease mechanisms are not well understood. A need remains for methods to treat these disorders.

### SUMMARY OF THE DISCLOSURE

In some embodiments, methods are disclosed for treating a subject with a cblC deficiency. These methods include selecting a human subject with the cblC deficiency; and administering about 5 mg to about 10 grams of MeCbl daily to the human subject.

In more embodiments, methods are disclosed for treating a fetus with a cblC deficiency. The methods include selecting a female human subject pregnant with the fetus that has the cblC deficiency; and administering about 5 mg to about 10 grams of MeCbl daily to the female human subject, in order to treat the cblC deficiency in the fetus.

The foregoing and other features and advantages of the invention will become more apparent from the following detailed description of several embodiments which proceeds with reference to the accompanying figures.

### BRIEF DESCRIPTION OF THE FIGURES

FIGS. 1A-1G. Production and characterization of the cblC (Mmachc) Deficiency Mouse Model. A) A portion of the exon 2 Mmachc cDNA sequence (NM_025962.3) (SEQ ID NO: 1), protein sequence (SEQ ID NO: 2) and variants created by TALEN genome editing are shown. The boxed region indicates predicted binding site of the TALENs. The cDNA sequence of de13 is SEQ ID NO: 3, and the encoded protein is SEQ ID NO: 4. The cDNA sequence of de12 is SEQ ID NO: 5, and the encoded protein is SEQ ID NO: 6. B) Photograph of Mmachc^{de13} litter at postnatal day 4 demonstrates that mutants are smaller and have less pigment/fur than their control littermates. At P28, Mmachc^{de13/de13} mutant shows severe growth impairment and also hypopigmented ears, tail and feet compared to its littermate. C) Survival of both Mmachc mice was poor with no mice living beyond 1 month Mmachc^{+/+} N=108, Mmachc^{+/de13} N=120, Mmachc^{de13/de13} N=66, Mmachc^{+/de12} N=81, Mmachc^{de12/de12} N=14. D) Mmachc^{de13/de13} were significantly growth impaired compared to control littermates at two weeks of age. E,F,G) Mmachc^{-/-} mice recapitulate the metabolic phenotype of cblC with elevated methylmalonic acid (E), homocysteine (F) and a trend toward low methionine (G). Mmachc^{de12/de12} were measured at P2 days and in all other mice, metabolite values were measured at P14-16 days. ****=p<0.0001.

FIGS. 2A-2F. Response to OHCbl treatment. Pregnant Mmachc^{+/de13} mice were treated weekly with 1 mg of OHCbl (prenatal OHCbl), and a cohort of mice continued to receive OHCbl every week (prenatal OHCbl+weekly). A) Improvement of survival of Mmachc^{de13/de13} was observed in the prenatal OHCbl group p<0.001 n=25 and prenatal OHCbl+weekly group p<0.0001 n=17 vs untreated mutants. Long term therapy with OHCbl was better than prenatal OHCbl alone p<0.05. B) Photograph shows that with long term OHCbl therapy, pigmentation differences in the tail and ears are improved. C) Relative weights of mutant mice at two weeks showed that both prenatal OHCbl p<0.0001 and prenatal OHCbl+weekly<0.01 n=9 improved the weight vs untreated but they did not reach control weights. D) Long term weight monitoring in both groups confirms that the mutants are smaller despite ongoing treatment (controls prenatal OHCbl n=19, controls prenatal OHCbl+weekly n=6, de13/de13 Prenatal OHCbl n=9, de13/de13 Pre & Weekly OHCbl n=4). E) Methylmalonic acid remained elevated vs controls and untreated mutants in both treatment groups at 7-13 months. F) Homocysteine remained elevated vs controls but was lower in both treatment groups at 7-13 months. *p<0.05, **p<0.01, ***p<0.001, ****p<0.0001.

FIGS. 3A-3F. Survival of prenatal OHCbl and/or MeCbl cobalamin treated Mmachc mice. Mmachc^{+/-} pregnant dams were treated weekly with OHCbl (1 mg), MeCbl (1 mg, approximately 7 mg/kg/day) or combination OHCbl/MeCbl (0.5 mg each) throughout gestation and weaning. A) Survival of the Mmachc^{de13/de13} mutants improved with prenatal OHCbl (median 109 days, p<0.001, n=25) and MeCbl (median 144 days, p<0.0001, n=7) vs untreated (median 5 days, n=66) with MeCbl treated showing better neonatal survival. B) Photograph showing improvement of hypopigmentation of the ear with prenatal MeCbl therapy in Mmachc^{de13/de13} mutants. C) Prenatally MeCbl treated mice displayed poor growth (p<0.05, controls n=4-13, Mmachc^{de13/de13} n=3-5. D)Survival of the Mmachc^{de12/de12} mutants improved with prenatal MeCbl (median 78 days, p<0.0001, n=22) and combination OHCbl/MeCbl (median 33 days, p<0.01, n=12) treatment compared to untreated (median 5 days, n=14). There was a trend towards improved survival with prenatal OHCbl therapy (median 25 days, p=0.0515, n=13). There was a significant difference in the survival of prenatal MeCbl vs prenatal OHCbl (p<0.05) and combination OHCbl/MeCbl (p<0.001). E) Weight of the prenatal MeCbl treated mice over time shows that the Mmachc^{de12/de12} mutants are still smaller than control littermates despite improved survival (n=6 controls, n=3 mutants). F) Photograph shows mutant mouse treated prenatally with MeCbl has more ear pigmentation than untreated mice.

FIGS. 4A-4D. Selected metabolites were measured the plasma of P0-P30 Mmachc^{de13} mice. A) Methylmalonic acid was elevated in untreated Mmachc^{de13/de13} mutants (p<0.0001 vs controls) and was decreased in Mmachc^{de13/de13} mice treated with weekly OHCbl (p<0.01) but not with prenatal OHCbl or prenatal MeCbl treatment. B,C) Both total methylcitrate, a metabolite related to MMA, and homocysteine were elevated in Mmachc^{de13/de13} mutants compared to controls (p<0.05 and p<0.0001 respectively). Treatments did not decrease total methylcitrate and homocysteine levels. D) Cystathionine, a downstream metabolite of homocysteine, increased with prenatal OHCbl treatment (p<0.05) and decreased to control levels with prenatal MeCbl treatment (p<0.01).

FIGS. 5A-5F. A) Survival curve of AAV treated Mmachc^{de13/de13} mice. All treatments vs untreated mutants were significant p<0.0001. Treatment with AAV9-coMMACHC and AAVrh10-Mmachc conferred long term survival with a single neonatal injection of 1E11 VG/pup. B) Relative weight of mutants at P14-16 showed that all treatments improved weights vs untreated Mmachc^{de13/de13} AAV9 gene therapy was equivalent to OHCbl. C) Photograph of wildtype and mutant mouse treated with AAV9 gene therapy showing improved weight and clinical appearance. D) Weight over time in the prenatal OHCbl+AAV9 treated mice vs mice treated with prenatal OHCbl and weekly OHCbl. Both groups of treated mice were smaller than controls but there was no significant different between the two treatments E) Mmachc^{de12} mice treated prenatally with MeCbl were received retroorbital injection of AAV9-coMMACHC. All three mice showed improved weight gain following the injection. E) Methylmalonic acid levels in the blood [MMA]were reduced at 7-13 months with prenatal OHCbl+AAVrh10 (182 μM; n=4) and prenatal OHCbl+AAV9 (137 μM; n=3) treatment, but not with prenatal+postnatal OHCbl (>1500 μM; n=4) treatment. F) There was no statistically significant difference in total homocysteine levels (tHCYS) at 7-13 months with prenatal OHCbl+AAVrh10 (22 μM; n=4) and prenatal OHCbl+AAV9 (21 μM; n=3) treatment, but not with prenatal+postnatal OHCbl (31 μM; n=4) treatment.

FIGS. 6A-6E. Pathological examination of untreated wild-type and Mmachc mice. A) Thinning of corpus callosum indicated by arrow and dilation of ventricles was observed in mutant mice at 1 month. B) The mutant mice do not exhibit the retinal degeneration seen in patients at 6 months of age. C) The Oil Red 0 staining indicated severe hepatic lipidosis in the Mmachc^{de13/de13} mutants. D) Macrovesicular lipidosis was observed in electron microscopy of the liver at 1 week. E) The testes of Mmachc^{de13/de13} mice at one month are hypoplastic with increased numbers of apoptotic round spermatid cells compared to littermate controls.

FIGS. 7A-7D. Embryonic phenotype of cblC. Embryos were removed at E18.5 from pregnant dams, weighed and photographed for measurements. A) Weight normalized to Mmachc and Mmachc littermates (controls) showed that both Mmachc^{de12/de12 and} Mmachc^{de13/de13} mutants weighed less than controls (controls 0.9989±0.01252, n=96, de12/de12 0.7336±0.03029, n=18, de13/de13 0.7705±0.0339, n=14) and prenatal OHCbl treatment did not improve the weight of the Mmachc^{de13/de13} mutants (de13/de13 OHCbl 0.8204±0.03641, n=11). B) Measurement of crown rump length also showed intrauterine growth retardation (IUGR) of both mutants and prenatal OHCbl did not rescue the IUGR. C) Measurement of anterioposterior abdominal dimension (APD) was also smaller in mutant mice and did not improved with prenatal OHCbl treatment. ****=p<0.0001. D) Photograph showing an Mmachc^{de13/de13} mutant with intrauterine growth retardation.

FIGS. 8A-8D. Selected metabolites were measured the plasma of P0-P30 Mmachc mice. A) Methylmalonic acid levels were elevated in untreated Mmachc^{de12/de12} and Mmachc^{de13/de13} mutants. Methylmalonic acid levels were decreased in Mmachc^{de13/de13} mice treated with weekly OHCbl (p<0.01) and AAV9-MMACHC gene therapy (p<0.001) but not with prenatal OHCbl or prenatal MeCbl treatment. B, C) Treatments did not decrease total methylcitrate and homocysteine levels although they remained elevated compared to control mice. D) The levels of cystathionine, a downstream metabolite of homocysteine, increased with prenatal OHCbl treatment (p<0.05) and decreased to control levels with prenatal MeCbl treatment (p<0.01).

FIGS. 9A-9D. Mouse embryonic fibroblast (MEF) studies. A) Both mutant MEF lines display decrease ¹⁴C propionate incorporation into protein compared to wildtype cell line (p<0.001). B) Total uptake of cobalamin (in pg/10⁶ cells) in MEF had decreased cobalamin uptake. C &D) Determination of MEF intracellular cobalamin distribution by supplementation of fibroblast media with [⁵⁷Co]CNCbl was performed to evaluate conversion to cobalamin derivatives. Both mutant cell lines exhibited adenosylcobalamin (AdoCbl) and MeCb deficiencies which is a hallmark of cblC patient fibroblasts.

FIG. 10. Vector design for the AAV constructs. Both vectors contain transcriptional control elements from the cytomegalovirus enhancer/chicken β-actin promoter. The AAV9 construct contains a codon optimized human MMACHC gene and the AAVrh10 construct contains the wildtype murine Mmachc gene.

FIGS. 11A-11D. A) Weight of E18.5 embryos normalized to control values. Prenatal MeCbl treatment improved the weight of the mutant embryos compared to untreated p=0.0004 (untreated 76.4% of controls vs MeCbl 89.4% of controls). Three mutants all from one litter were outliers (*). B) H&E staining of lung in E18.5 untreated mutant embryos shows decreased alveolar spaces and alveolar septa with less squamous differentiation (top right). The alveolar spaces are present in prenatal MeCbl treated embryos (bottom right). C) H&E staining of liver in E18.5 untreated mutant embryos shows lack of cytoplasmic clearing in hepatocytes possibly due to decreased glycogen stores (top right). Cytoplasmic clearing is restored with prenatal MeCbl treatment (bottom right). D) H&E staining of brown adipose tissue in E18.5 untreated mutant embryos shows hypoplasia brown fat and adipocytes that lack vesicles containing lipids (top right) which is ameliorated with prenatal MeCbl therapy (bottom right).

### SEQUENCE LISTING

The nucleic and amino acid sequences listed are shown using standard letter abbreviations for nucleotide bases, and one or three letter code for amino acids, as defined in 37 C.F.R. 1.822. Only one strand of each nucleic acid sequence is shown, but the complementary strand is understood as included by any reference to the displayed strand.

SEQ ID NO: 1 is a portion of exon 2 of the Mmachc cDNA sequence (GENBANK® Accession No. NM_025962.3, Oct. 1, 2020, incorporated herein by reference).

SEQ ID NO: 2 is the protein sequence encoded by this portion of exon 2 of the Mmachc cDNA sequence (GENBANK® Accession No. NM_025962.3, Oct. 1, 2020, incorporated herein by reference).

SEQ ID NO: 3 is the cDNA sequence of the corresponding portion of exon 2 of de13.

SEQ ID NO: 4 is the protein sequence encoded by this portion of exon 2 of de13.

SEQ ID NO: 5 is the cDNA sequence of the corresponding portion of exon 2 of de12.

SEQ ID NO: 6 is the protein sequence encoded by this portion of exon 2 of de12.

### DETAILED DESCRIPTION OF SEVERAL EMBODIMENTS

CblC deficiency is the most common inborn error of intracellular cobalamin metabolism (Lerner-Ellis et al. Nat. Genet., 38: 93-100 (2006)) and is caused by mutations in MMACHC (GENBANK® Accession No. NM_015506.2, Oct. 1, 2020, incorporated herein by reference), a gene responsible for the processing and trafficking of intracellular cobalamin. Mutations in MMACHC impair the activity of two cobalamin-dependent enzymes: methylmalonyl-CoA mutase (MUT) and methionine synthase (MTR). MMACHC transports and processes intracellular cobalamin into its two active co factors, 5′-adenosyl cobalamin and MeCbl, necessary for the enzymatic reactions of MUT and MTR, respectively. Patients display methylmalonic acidemia, hyperhomocysteinemia, and hypomethionemia and variably manifest intrauterine growth retardation, anemia, heart defects, failure to thrive, white matter disease, neuropathy, neurocognitive impairment, and a progressive maculopathy, pigmentary retinopathy, and retinal degeneration that causes blindness, despite standard of care metabolic therapy (Carrillo-Carrasco et al., J. Inherit. Metab. Dis., 35: 103-14 (2012)).

In the United States, cblC is often diagnosed based on newborn screening. While the true prevalence of the disorders of intracellular cobalamin metabolism is unknown, the historical incidence of cblC has been estimated at 1:200,000 births with about 400 cases reported in the literature; data from newborn screening suggested a higher incidence closer to 1:100,000 in New York state and 1:60,000 in California, where an incidence of 1:37,000 was estimated in the Hispanic population. In one study of a Chinese population in Shangong province, it was claimed that 1:3920 births were affected (Han et al., China, Brain Dev., pii: S0387-7604(15)00228-4 (2015)). Disclosed herein a method for treating a cobalamin disorder, such as, but not limited to cblC type, using a high dose of MeCbl.

It is disclosed herein that during the course of study of Mmachc knock out mice, a prominent and severe in utero and neonatal phenotype became apparent, first manifesting as significant skewing in the numbers of expected: observed Mmachc mutants at birth. However, unexpectedly in the Mmachc^{de12} mice, prenatal treatment with MeCbl or a combination of MeCbl and OHCbl, but not OHCbl alone, restored genotype ratios to normal. Furthermore, prenatal MeCbl dramatically improved the neonatal survival of both mutants and was superior to prenatal OHCbl. In addition, improved survival was accompanied by a biomarker response (normalization of cystathionine) that is specific to MeCbl and not OHCbl therapy. Thus, in one embodiment, the disclosed method results in a change in cytathoinine. Improvement in pigmentation was also detected using prenatal MeCbl treatment as compared to OHCbl where the mutants had minimal pigmentation changes in their fur, ears and tail and were difficult to distinguish from control littermates

### Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Unless otherwise indicated, “about” indicates within five percent (5%).

Unless explained otherwise, all technical and scientific terms used herein have the same meaning as commonly understood to one of ordinary skill in the art to which this disclosure belongs. All GENBANK® Accession Nos. listed herein are incorporated by reference in their entirety as available on Oct. 1, 2020, unless indicated otherwise. Although methods and materials similar or equivalent to those described herein can be used in the practice or testing of the present disclosure, suitable methods and materials are described below. The materials, methods, and examples are illustrative only and not intended to be limiting.

Administration: The introduction of an agent, such as MeCbl, or a combination of MeCbl and OHCbl, into a subject by a chosen route. Administration can be local or systemic. For example, if the chosen route is intravenous, the agent is administered by introducing the composition into a vein of the subject. Exemplary routes of administration include, but are not limited to, oral, injection (such as subcutaneous, intramuscular, intradermal, intraperitoneal, and intravenous), sublingual, intrathecal, subretinal, intravitreous, rectal, transdermal (for example, topical), intranasal, vaginal, and inhalation routes.

Adult, Child, Infant, and Fetus: A human “adult” subject is greater than about 18 years of age, and has completed puberty, such as a subject in their 20s, 30s, 40s, 50s or 60s. A human “child” is 17 years or less of age. This includes “teenagers” who are going through puberty, and “pre-pubescent” children. A human infant is about 1 year of age or less. A human “fetus” is in utero, in a pregnant subject. A human “newborn” or “neonate” is one month or less of age.

Co-Administration: Administration of two or more compositions to a subject together, which includes administration at about the same time or within a certain specific or desired time.

Cobalamin C (cblC) deficiency: An inborn error of intracellular cobalamin metabolism (Lerner-Ellis et al. Nat. Genet., 38: 93-100 (2006)) that is caused by mutations in MMACHC (GENBANK® Accession No. NM_0 15506.2, Oct. 1, 2020, incorporated herein by reference), a gene responsible for the processing and trafficking of intracellular cobalamin. Pathogenic variants in MMACHC impair the activity of two cobalamin-dependent enzymes: methylmalonyl-CoA mutase (MUT) and methionine synthase (MTR). MMACHC transports and processes intracellular cobalamin into its two active cofactors, 5′-adenosyl cobalamin and MeCbl, necessary for the enzymatic reactions of MUT and MTR, respectively. cblC can also be caused by pathogenic variants in a neighboring gene PRDX1 (epi-cblC) and transcriptional regulators of MMACHC (HCFC1, THAP11, ZNF143). Affected individuals display methylmalonic acidemia, hyperhomocysteinemia, and hypomethionemia and variably manifest intrauterine growth retardation, anemia, heart defects, failure to thrive, hemolytic uremic syndrome, thrombotic microangiopathy, white matter disease, seizures, hydrocephalus, neuropathy, neurocognitive impairment, and a progressive maculopathy, pigmentary retinopathy, and retinal degeneration that causes blindness (Carrillo-Carrasco et al., J. Inherit. Metab. Dis., 35: 103-14 (2012)).

The diagnosis of a disorder of intracellular cobalamin metabolism is based on clinical, biochemical, and molecular genetic data. Evaluation of the methylmalonic acid (MMA) level in urine and blood and plasma total homocysteine (tHcy) level are the mainstays of biochemical testing. Diagnosis is confirmed by identification of biallelic pathogenic variants in one of the following genes (associated complementation groups indicated in parentheses): MMACHC (cblC), PRDX1(epi-cblC), MMADHC (cblD-combined and cblD-homocystinuria), MTRR (cblE), LMBRD1 (cblF), MTR (cblG), ABCD4 (cblf), THAP11(cblX-like), ZNF143(cblX-like), or a hemizygous variant in HCFC1 (cblX). HCFC1, THAP11 and ZNF143 can be classified as cblC using complementation studies, due to dysregulation of MMACHC.

Left ventricular non-compaction, congenital heart defects, dilated cardiomyopathy and heart failure can be seen in individuals with this deficiency. Individuals with cblC and other cobalamin disorders are at risk of acute or chronic kidney injury due to thrombotic microangiopathy resulting in thrombocytopenia, microangiopathic hemolytic anemia and hemolytic uremic syndrome.

Control: A reference standard. In some embodiments, the control is a negative control sample obtained from a healthy patient. In other embodiments, the control is a reference sample, or a reference test, before treatment of the same subject using the methods disclosed herein.

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Diagnosis: The process of identifying a disease by its signs, symptoms and results of various tests. The conclusion reached through that process is also called “a diagnosis.” Forms of testing commonly performed include blood tests, medical imaging, urinalysis, and biopsy. Diagnosis can also be made on the basis of genetic tests, such as for specific genes and nucleotide sequences or enzymatic and biochemical studies (including ¹⁴C propionate and ¹⁴C methyl-THF incorporation and B₁₂ responsiveness, complementation analysis, and cobalamin distribution assays) in skin biopsy-derived fibroblasts.

Homocystinuria: Homocystinuria is defined as the presence of homocystine in the urine following urine amino acid analysis. Homocystinuria can be caused by deficiency of vitamins B₆, B₁₂, or folate. Hereditary causes of homocystinuria include the hereditary cobalamin disorders, due to the inability to synthesize methylcobalamin which is required for the remethylation of homocysteine to form methionine. Disorders that feature methylcobalamin deficiency include: cblC, epi-cblC, cblD, cblE, cblF, cblG, cblf, cblX. Homocystinuria can also be caused by cystathionine beta synthase (CBS), an autosomal recessive disorder of the metabolism of homocysteine. The homocystinuria seen in disorders of intracellular cobalamin metabolism is associated with low/normal methionine in contrast to the homocystinuria seen in cystathionine beta-synthase deficiency, which is associated with high methionine.

Intrauterine Growth Retardation (IUGR): Less than 10 percent of predicted fetal weight for gestational age. IUGR manifests as a continuum ranging from asymmetry (early stages) to symmetry (late stages). Symmetric IUGR, including non-head sparing IUGR, refers to fetuses with equally poor growth velocity of the head, the abdomen and the long bones. Asymmetric IUGR refers to infants whose head and long bones are spared compared with their abdomen and viscera.

IUGR is frequently detected in a pregnancy with a less-than-expected third-trimester weight gain (100 to 200 g (3.5 to 7 oz) per week) or as an incidental finding on ultrasound examination when fetal measurements are smaller than expected for gestational age.

Macular Disease: An eye disease that progressively destroys the macula, which is the central portion of the retina, impairing central vision. Macular disease includes macular atrophy, macular coloboma, and blindness due to degeneration of the macula.

Methylmalonic acidemia: Elevated methylmalonic acid in the blood, serum or plasma. Methylmalonic acidemia can be caused by vitamin B12 deficiency. The term is also used to describe a group of inherited disorders caused by complete or partial deficiency of the enzyme methylmalonyl-CoA mutase (mut⁰ enzymatic subtype or mut⁻ enzymatic subtype, respectively), a defect in the transport or synthesis of its cofactor, adenosyl-cobalamin (cblA, cblB, cblC, cblD, cblF, cblf), or deficiency of the enzyme methylmalonyl-CoA epimerase. See Manoli et al., “Isolated Methylmalonic Acidemia,” Gene Reviews [Internet], Dec. 1, 2016, incorporated herein by reference.

Diagnosis of methylmalonic acidemia can be detected using analysis of organic acids in plasma, serum and/or urine by gas-liquid chromatography and mass spectrometry. Establishing the specific subtype of methylmalonic acidemia requires cellular biochemical studies (including ¹⁴C propionate incorporation and B₁₂ responsiveness, complementation analysis, and cobalamin distribution assays) and molecular genetic testing. The finding of biallelic pathogenic variants in one of the following genes is associated with methylmalonic acidemia (MMUT, MMAA, MMAB, MCEE, MMADHC, MMACHC, PRDX1, LMBRD1, ABCD4) or hemizygous variant in HCFC1, with confirmation of genetics of the parents, can establish the diagnosis.

Neurocognitive Function: Cognitive functions closely linked to the function of particular areas, neural pathways, or cortical networks in the brain, ultimately served by the substrate of the brain's neurological matrix (i.e. at the cellular and molecular level), see the DSMV-5 for tests of neurocognitive function. These tests may include Vineland Adaptive Behavior Scales, Differential Ability Scales, Mullen Scales of Early Learning, Wechsler Intelligence Scale for Children and Wechsler Adult Intelligence Scale, among others.

Subjects with cblC can have a variety of cognitive defects which are related to disease severity and treatment onset but can continue to worsen. A subject can have developmental delays, moderate to severe intellectual impairment, autism or autism spectrum disorder, and declines in attention and executive function. Without treatment, subjects can have progressive encephalopathy with regression, deterioration in school or work performance, behavioral and personality changes that may result in dementia, psychosis, episodes of acute mental confusion, lethargy and seizures.

Neurological complications: Subjects with cblC can present with neurological impairments at all levels of the nervous system, including the cerebral cortex (encephalopathy, epilepsy, dyspraxia), pyramidal tracts (spasticity), basal ganglia and cerebellum (causing abnormal movement), and peripheral nerves. A dysfunction of peripheral nerves, typically causing numbness or weakness and abnormal sweating. These are associated with abnormal brain imaging findings, including cerebral and cerebellar atrophy, with white matter thinning/leukodystrophy, corpus callosum dysgenesis and hydrocephalus. The hyperhomocysteinemia is a high risk factor for thromboembolic events and some patients can present with severe CNS strokes.

Retinal Degeneration: A pathological impairment of the retina, such as in the rod, cones or retinal pigment epithelial cells in the retina, that impairs vision.

Seizures: A sudden, uncontrolled electrical disturbance in the brain. These include absence seizures, myoclonic, tonic-clonic seizures, infantile spasms, and focal seizures.

Vitamin B₁₂: Also called “cobalamin” which includes the isoforms OHCbl, cyanocobalamin, MeCbl or adenosylcobalamin. The active forms in vivo are MeCbl and 5′-deoxyadenosylcobalamin, which are necessary for methylmalonyl-CoA, homocysteine and folic acid metabolism. MeCbl also catalyzes the demethylation of a folate cofactor which is involved in DNA synthesis. A lack of demethylation may result in folic acid deficiency. 5′-deoxyadenosylcobalamin is the coenzyme for the conversion of methylmalonyl-CoA to succinyl-CoA, which plays a role in the citric acid cycle. Cobalamin, along with pyridoxine and folic acid, also are implicated in the proper metabolism of homocysteine, and required for methionine synthesis, a precursor of S-adenosylmethionione which is important for methylation. In specific embodiments, vitamin B12 may be in one or more of the forms of cobalamin, MeCbl, 5′-deoxyadenosylcobalamin (adenosylcobalamin or cobamamide), cyanocobalamin, hydroxocobalamin, and aquacobalamin.

Composition or a Formulation: A product comprising the specified active ingredients in the specified amounts, as well as any product which results, directly or indirectly, from the combination of the specified active ingredients in the specified amounts. Such term is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the compositions encompass any composition made by mixing any active compound, such as a cobalamin, for example, MeCbl, and a pharmaceutically acceptable carrier.

Inert Ingredients: Components such as pharmaceutically acceptable carriers, adjuvant, diluents or excipients, etc., that must be compatible with the other ingredients of the formulation and not deleterious to the recipient thereof.

Pharmaceutically acceptable carriers: The pharmaceutically acceptable carriers of use are conventional. Remington's Pharmaceutical Sciences, by E. W. Martin, Mack Publishing Co., Easton, PA, 15th Edition (1975), describes compositions and formulations suitable for pharmaceutical delivery of the therapeutic agents to treat a cblC deficiency are disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

A “therapeutically effective amount” is a quantity of a composition to achieve a desired effect in a subject being treated. For instance, this can be the amount of methylcobalamin (MeCbl), or a combination of MeCbl and hydroxocobalamin (OHcbl), necessary to reduce symptoms of a cblC deficiency. When administered to a subject, a dosage will generally be used that will achieve target tissue concentrations that has been shown to achieve an in vitro effect.

Subject: Living Multi-Cellular Vertebrate Organism, a Category that Includes Human

Additional terms commonly used in molecular genetics can be found in Krebs et al. (eds.), Lewin's genes XII, published by Jones & Bartlett Learning, 2017, The Encyclopedia of Cell Biology and Molecular Medicine, published by Wiley-VCH in 16 volumes, 2008; and other similar references.

### Methods of Treatment

Disclosed herein are methods for treating a cblC deficiency in a subject, such as a human subject. The methods include selecting a human subject with the cblC deficiency and administering about 5 milligram (mg) to about 10 grams of MeCbl daily to the human subject.

In some embodiments, the methods include administrating about 5 mg to about 10 grams of MeCbl daily to the human subject. The method can include administering about 10 mg to about 10 grams of MeCbl daily to the human subject, or about 5 mg to about 5 grams of MeCbl daily to the human subject. The use of other doses within these ranges is also contemplated.

The method can include administering about 5 mg to about 50 mg daily to the human subject, such as about 10 to about 40 mg daily to the human subject. The method can include administering about 5 to about 10, 15, 20, 25, 30, 35, 40, 45 or 50 mg of MeCbl daily to the human subject. The method can include administering about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg of MeCbl daily to the human subject. The use of other doses within these ranges is also contemplated.

The method can include administering about 1 gram to about 8 grams of MeCbl daily to the human subject, about 2 to about 5 grams daily to the human subject, about 2 to about 10 grams, or about 5 to about 10 grams daily to the human subject. The method can include administering about 1, 2, 3, 4, 5, 6, 7, 8, 9, or 10 grams per day. The use of other doses within these ranges is also contemplated. In some embodiments, the MeCbl is administered intravenously.

The human subject can be an infant, such as a newborn, a child or an adult. In some embodiments, the MeCbl is administered to the human subject at a dose of about 0.3 to about 5.5 mg/kg/day. In other embodiments, the MeCbl is administered to the human subject at a dose of about 0.3 to about 1.4 mg/kg/day. In more embodiments, the MeCbl is administered to the human subject at a dose of about 0.5 to about 4.0 mg/kg/day. In further embodiments, the MeCbl is administered to the human subject at a dose of about 25 mg/kg/day to about 100 mg/kg/day. The MeCbl can be administered at a dose of about 0.05, 0.4, 0.5, 0.6, 0.7, 0.8, 0.9, 1.0, 1.1, 1.2 or 1.3 mg/kg/day. The MeCbl can be administered at a dose of about 0.5, 1.0, 1.5, 2.0, 2.5, 3.0, 3.5, 4.0, 4.5 or 5.0 mg/kg/day. The MeCbl can be administered at a dose of about 5, 10, 15, 20, 25, 30 35, 40, 45, 50, 55, 60, 65, 60, 75, 80, 85, 90 or 100 mg/kg/day. The use of other doses within these ranges is also contemplated.

The MeCbl can be administered by any route, including, but not limited to intravenous, intramuscular, or subcutaneous administration. The MeCbl can be administered by other routes, such as, but not limited to, oral or intranasal administration. Exemplary routes of administration include, but are not limited to, oral, injection (such as subcutaneous, intramuscular, intradermal, intraperitoneal, and intravenous), sublingual, intrathecal, subretinal, intravitreous, rectal, transdermal (for example, topical), intranasal, vaginal, and inhalation routes.

In some embodiments, the method also includes administering to the subject a therapeutically effective amount of OHCbl. In some embodiments, the methods include administrating about 2 mg to about 10 mg, such as 5 mg to about 10 grams of OHCbl daily to the human subject. The method can include administering about 10 mg to about 10 grams of OHCbl daily to the human subject, or about 5 mg to about 5 grams of OHCbl daily to the human subject. The use of other doses within these ranges is also contemplated.

The method can include administering about 5 mg to about 50 mg daily to the human subject, such as about 10 to about 40 mg daily to the human subject. The method can include administering about 5 to about 10, 15, 20, 25, 30, 35, 40, 45 or 50 mg of OHCbl daily to the human subject. The method can include administering about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg of OHCbl daily to the human subject. The use of other doses within these ranges is also contemplated.

The method can include administering about 1 gram to about 8 grams of OHCbl daily to the human subject, about 2 to about 10 grams daily to the human subject, about 2 to about 5 grams daily to the human subject, or about 5 to about 10 grams daily to the human subject. The method can include administering about 1, 2, 3, 4, 5, 6, 7, 8, 9, or 10 grams per day. The use of other doses within these ranges is also contemplated. In some embodiments, the OHCbl is administered intravenously.

The OHCbl can be administered by any route, including, but not limited to intravenous, intramuscular, or subcutaneous administration. The OHCbl can be administered by other routes, such as, but not limited to, oral or intranasal administration. Exemplary routes of administration include, but are not limited to, oral, injection (such as subcutaneous, intramuscular, intradermal, intraperitoneal, and intravenous), sublingual, intrathecal, subretinal, intra-vitreous, rectal, transdermal (for example, topical), intranasal, vaginal, and inhalation routes.

The MeCbl and the OHCbl can be administered together, in a single dose and/or at the same time, or can be administered sequentially. For example, the OHCbl can be administered within about 5 to about 120 minutes of the MeCbl, such as about 5 to about 2 hours such as about 5, 10, 15, 20, 25, 30, 35, 40, 45, 50, 55 or 60 minutes. The OHCbl can be administered within about 5 minutes, within about 10 minutes, within about 20 minutes, or within about 30 minutes of the MeCbl. In some embodiments the ratio of MeCbl to OHCbl is about 5:1, 4:1, 3:1, 2:1, 1:1, 1:2, 1:3, 1:4, or 1:5.

In the presently disclosed methods, the human subject can also be administered a therapeutically effective amount of betaine, folate and/or folinic acid. In some embodiments, a subject with elevated total plasma homocysteine (tHcy) is administered betaine (for example, at about 250 mg/kg/day) and folate or folinic acid. Betaine can be administered in divided doses, such as 3 to 4 doses per day. Without being bound by theory, betaine dose can be titrated to response while monitoring tHcy and plasma methionine. In specific, nonlimiting examples, betaine can be administered at about 250 mg/kg/day. In other non-limiting examples, folinic acid can be administered at about 5 to about 30 mg/day. In further non-limiting examples, folate can be administered at about 10 mg/day.

The nomenclature for inherited disorders of intracellular cobalamin metabolism is based on cellular complementation analysis that defines cobalamin groups A-J (cblA-cblJ). The name of each disorder is prefixed with “cbl” (for cobalamin) followed by a unique capital letter for its complementation group determined by somatic cell analysis (e.g., cblC represents complementation group C). cblA, cblB, and cblD-MMA cause isolated methylmalonic aciduria whereas cblC, cblD-homocytinuria, cblF, cblf cause a combined methylmalonic aciduria and hyperhomocysteinemia and cblD-homocytinuria, cblE and cblG cause isolated homocystinuria. These disorders of intracellular cobalamin metabolism are inherited in an autosomal recessive manner except for cblX (associated with pathogenic variants in HCFC1), which is inherited in an X-linked manner A subject can be selected for treatment that has a pathogenic variant in one of the following genes (associated complementation groups indicated in parentheses): MMACHC (cblC), PRDX1 (epi-cblC) MMADHC (cblD-combined and cblD-homocystinuria), MTRR (cblE), LMBRD1 (cblF), MTR (cblG), ABCD4 (cblf), THAP11 (cblX-like), ZNF143(cblX-like), or a hemizygous variant in HCFC1 (cblX). HCFC1, THAP11 and ZNF143 can be classified as cblC using complementation studies, due to dysregulation of MMACHC. In some embodiments, a subject can be selected for treatment that has cblC pathogenic variant. The age of initial presentation of cblC and other cobalamin disorders spans a wide range. See Sloan et al., “Disorders of Intracellular Cobalamin Metabolism,” in GeneReviews [Internet], updated on Sep. 6, 2018, available on the internet at ncbi.nlm.nih.gov/books/NBK1328/, incorporated herein by reference.

A subject can be selected for treatment that has symptoms of a cblC deficiency. In some embodiments, a subject can be selected for treatment that has cblC pathogenic variant. In some embodiments the subject is a newborn, and can have microcephaly, poor feeding, and/or encephalopathy. In further embodiments, the subject is an infant, and can have poor feeding and slow growth, neurologic abnormality, and/or hemolytic uremic syndrome (HUS). In yet other embodiments, the subject is a child, and can have poor growth, progressive microcephaly, cytopenia (including megaloblastic anemia), global developmental delay, encephalopathy, and/or neurologic signs such as hypotonia and seizures. In some embodiments, the subject is an adolescent or an adult, and can have neuropsychiatric symptoms, progressive cognitive decline, thromboembolic complications, and/or subacute combined degeneration of the spinal cord, or dilated cardiomyopathy and heart failure, or acute or chronic kidney injury due to thrombotic microangiopathy resulting in thrombocytopenia, microangiopathic hemolytic anemia and hemolytic uremic syndrome. In some embodiments, one or more of these symptoms is improved following treatment.

A subject can be selected for treatment based on laboratory tests. In some embodiments, a subject can be selected for treatment that has laboratory findings of macrocytic anemia with normal B₁₂ levels, thrombocytopenia, and/or neutropenia. In further embodiments, the subject can be selected for treatment that has laboratory findings of hyperammonemia and/or metabolic acidosis in infancy.

In some embodiments, a subject is selected for treatment that has methylmalonic acidemia. In other embodiments, a subject is selected for treatment that has homocystinuria. In more embodiments, a subject is selected for treatment that has combined methylmalonic acidemia and homocystinuria.

The disclosed methods result in improvement of at least one sign or symptom of the cblC deficiency. In some embodiments, the human subject has macular disease, and retinal function is improved following treatment. In other embodiments, the human subject has impaired neurocognitive function, and a neurocognitive outcome is improved following treatment. In yet other embodiments, the human subject has seizures, and the seizures are improved following treatment. In further embodiments, the human subject has neuropathy, and this neuropathy is improved following treatment. In more embodiments, the subject has improved kidney function following treatment. In yet other embodiments, the subject has cardiomyopathy, which is improved following treatment. In yet other embodiments, the subject has leukodystrophy, which is improved following treatment. In some embodiments, the subject has autism, which is improved following treatment.

Also disclosed herein is a method of treating a fetus with a cobalamin C (cblC) deficiency. The methods include selecting a female human subject pregnant with the fetus that has the cblC deficiency; and administering about 5 mg to about 10 grams of MeCbl daily to the female human subject, thereby treating the cblC deficiency in the fetus. In some embodiments, the female human subject can be in the second or third trimester of pregnancy.

In some embodiments, the methods include administrating about 5 mg to about 10 grams of MeCbl daily to the female human subject. The method can include administering about 10 mg to about 10 grams of MeCbl daily to the female human subject, or about 5 mg to about 5 grams of MeCbl daily to the female human subject. The use of other doses within these ranges is also contemplated.

The method can include administering about 5 mg to about 50 mg daily to the female human subject, such as about 10 to about 40 mg daily to the human subject. The method can include administering about 5 to about 10, 15, 20, 25, 30, 35, 40, 45 or 50 mg of MeCbl daily to the female human subject. The method can include administering about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg of MeCbl daily to the female human subject. The use of other doses within these ranges is also contemplated.

The method can include administering about 1 gram to about 8 grams of MeCbl daily to the female human subject, about 2 to about 10 grams daily to the female human subject, about 2 to about 5 grams daily to the female human subject, or about 5 to about 10 grams daily to the female human subject. The method can include administering about 1, 2, 3, 4, 5, 6, 7, 8, 9, or 10 grams per day. The use of other doses within these ranges is also contemplated. In some embodiments, the MeCbl is administered intravenously.

The MeCbl can be administered by any route, including, but not limited to intravenous, intramuscular, or subcutaneous administration. The MeCbl can be administered by other routes, such as, but not limited to, oral or intranasal administration. Exemplary routes of administration include, but are not limited to, oral, injection (such as subcutaneous, intramuscular, intradermal, intraperitoneal, and intravenous), sublingual, intrathecal, subretinal, intravitreous, rectal, transdermal (for example, topical), intranasal, vaginal, and inhalation routes.

In some embodiments, the MeCbl is administered to the female human subject at a dose of about 0.3 to about 5.5 mg/kg/day. In other embodiments, the MeCbl is administered to the female human subject at a dose of about 0.3 to about 1.4 mg/kg/day. In more embodiments, the MeCbl is administered to the female human subject at a dose of about 0.5 to about 4.0 mg/kg/day. In further embodiments, the MeCbl is administered to the female human subject at a dose of about 25 mg/kg/day to about 100 mg/kg/day. The MeCbl can be administered at a dose of about 0.05, 0.4, 0.5, 0.6, 0.7, 0.8, 0.9, 1.0, 1.1, 1.2 or 1.3 mg/kg/day. The MeCbl can be administered at a dose of about 0.5, 1.0, 1.5, 2.0, 2.5, 3.0, 3.5, 4.0, 4.5 or 5.0 mg/kg/day. The MeCbl can be administered at a dose of about 5, 10, 15, 20, 25, 30 35, 40, 45, 50, 55, 60, 65, 60, 75, 80, 85, 90 or 100 mg/kg/day. The use of other doses within these ranges is also contemplated.

The MeCbl can be administered by any route, including, but not limited to intravenous, intramuscular, or subcutaneous administration. The MeCbl can be administered by other routes, such as, but not limited to, oral or intranasal administration. Exemplary routes of administration include, but are not limited to, oral, injection (such as subcutaneous, intramuscular, intradermal, intraperitoneal, and intravenous), sublingual, intrathecal, subretinal, intravitreous, rectal, transdermal (for example, topical), intranasal, vaginal, and inhalation routes.

In some embodiments, the method also includes administering to the female human subject a therapeutically effective amount of OHCbl. In some embodiments, the methods include administrating about 2 mg to about 10 mg, such as about 5 mg to about 10 grams of OHCbl daily to the female human subject. The method can include administering about 10 mg to about 10 grams of OHCbl daily to the female human subject, or about 5 mg to about 5 grams of OHCbl daily to the female human subject. The use of other doses within these ranges is also contemplated.

The method can include administering about 5 mg to about 50 mg daily to the female human subject, such as about 10 to about 40 mg daily to the human subject. The method can include administering about 5 to about 10, 15, 20, 25, 30, 35, 40, 45 or 50 mg of OHCbl daily to the female human subject. The method can include administering about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg of OHCbl daily to the female human subject. The use of other doses within these ranges is also contemplated.

The method can include administering about 1 gram to about 8 grams of OHCbl daily to the female human subject, about 2 to about 5 grams daily to the human subject, or about 5 to about 10 grams daily to the female human subject. The method can include administering about 1, 2, 3, 4, 5, 6, 7, 8, 9, or 10 grams per day. The use of other doses within these ranges is also contemplated. In some embodiments, the OHCbl is administered intravenously.

The OHCbl can be administered by any route, including, but not limited to intravenous, intramuscular, or subcutaneous administration. The OHCbl can be administered by other routes, such as, but not limited to, oral or intranasal administration. Exemplary routes of administration include, but are not limited to, oral, injection (such as subcutaneous, intramuscular, intradermal, intraperitoneal, and intravenous), sublingual, intrathecal, subretinal, intra-vitreous, rectal, transdermal (for example, topical), intranasal, vaginal, and inhalation routes.

The MeCbl and the OHCbl can be administered together, in a single dose and/or at the same time, or can be administered sequentially. For example, the OHCbl can be administered within about 5 to about 120 minutes of the MeCbl, such as about 5 to about 2 hours such as about 5, 10, 15, 20, 25, 30, 35, 40, 45, 50, 55 or 60 minutes. The OHCbl can be administered within about 5 minutes, within about 10 minutes, within about 20 minutes, or within about 30 minutes of the MeCbl. In some embodiments the ratio of MeCbl to OHCbl is about 5:1, 4:1, 3:1, 2:1, 1:1, 1:2, 1:3, 1:4, or 1:5.

The fetus can have a pathogenic variant in one of the following genes (associated complementation groups indicated in parentheses): MMACHC (cblC), PRDX1 (epi-cblC), MMADHC (cblD-combined and cblD-homocystinuria), MTRR (cblE), LMBRD1 (cblF), MTR (cblG), ABCD4 (cblf), THAP11 (cblX-like), ZNF143(cblX-like), or a hemizygous variant in HCFC1 (cblX). In some embodiments, the fetus has cblC pathogenic variant. The fetus can have a pathogenic variant in one or more of the genes listed above. In more embodiments the fetus has in utero presentation of nonimmune hydrops, cardiomyopathy, and/or intrauterine growth retardation.

In the presently disclosed methods, the female human subject can also be administered a therapeutically effective amount of betaine, folate and/or folinic acid. Betaine can be administered in divided doses, such as 3 to 4 doses per day. In specific, nonlimiting examples, betaine can be administered at about 250 mg/kg/day. In other non-limiting examples, folinic acid can be administered at about 5 to about 30 mg/day. In further non-limiting examples, folate can be administered at about 10 mg/day.

In some embodiments, the fetus has intrauterine growth retardation (IUGR), and wherein growth of the fetus is improved following administration to the human subject. In further embodiments, wherein one or more of ocular function, macular function, cardiac function, brain growth, neurocognitive function or hydrocephalus is improved in the fetus following birth as compared to a control. In more embodiments, one or more of ocular function, macular function, cardiac function, renal function, thrombotic microangiopathy, seizures, neuropathy, kidney function, brain growth, neurocognitive function or hydrocephalus is improved following birth.

In some embodiments, the methods include performing at least one laboratory test. The test can be performed on a human subject with a cblC deficiency, at any time after birth. The test can be performed on an infant, a child or an adult with a cblC deficiency. The test can be performed following birth of a fetus, such as on a newborn or an infant.

In some embodiments, the method includes performing a urine organic acid (UOA) analysis to screen for elevation of the level of methylmalonic acid (MMA) in the subject prior to treatment, and/or a reduction in the levels of methylmalonic acid following treatment. An increase in secondary metabolites such as 3-hydroxypropionate, methylcitrate, and tiglylglycine may be seen transiently in symptomatic individuals. These can also be improved following treatment. In more embodiments, the method includes a serum methylmalonic acid analysis to screen for elevation of [MMA] prior to treatment, and/or a reduction in [MMA] following treatment. In other embodiments, the method includes performing a total plasma homocysteine (tHcy) analysis to screen for elevation of tHcy prior to treatment, and/or a reduction in tHcy following treatment. In more embodiments, the method includes a plasma amino acid (PAA) analysis. Without being bound by theory, hypomethioninemia, seen in disorders with defective MeCbl synthesis, helps differentiate disorders of intracellular cobalamin metabolism from other causes of homocystinuria, such as cystathionine beta-synthase deficiency. In further embodiments, the method includes a PAA analysis to screen for hypomethioninemia, or an improvement (increase) following treatment. In yet other embodiments, the method includes a PAA analysis to screen for hyperhomocysteinemia and mixed disulfides, or an improvement (decrease) following treatment. In more embodiments, the method includes gas chromatography mass spectrometry analysis to detect cystathionine or an improvement (decrease) following treatment. In further embodiments, the method includes assessing brain MR spectroscopy as an out parameter. Other metabolites such as S-adenosylmethionine, choline, creatinine, creatine, glycine and cysteine may also be measured. In more embodiments, the method includes brain MR spectroscopy as outcome parameter to quantify choline, creatine, N-acetylaspartate and other metabolites in the brain. In some embodiments, the method includes a serum vitamin B12 analysis. In more embodiments, the method includes performing a plasma acylcarnitine analysis to detect elevation of propionylcarnitine (C3) or confirm the elevated propionylcarnitine following newborn screening or confirm an improvement (decrease) following treatment. Any combination of these analysis can be performed. The method can include measurement of cystathionine. Metabolite concentrations in disorders of intracellular cobalamin metabolism are known, see for example Sloan et al., “Disorders of Intracellular Cobalamin Metabolism,” in GeneReviews [Internet], updated on Sep. 6, 2018, available on the internet at ncbi.nlm.nih.gov/books/NBK1328/, incorporated herein by reference.

In other embodiments, the method includes performing a test to evaluate ocular function, macular function (optical coherence tomography, electroretinogram, visual evoked potentials), cardiac function, brain growth and brain biochemistry by CSF amino acids, and brain MRI and MR spectroscopy parameters (choline, creatine, N-acetylaspartate), seizure activity (electroencephalogram), neuropathy (sweat test, nerve conduction studies), neurocognitive function and hydrocephalus. In some embodiments, the disclosed methods result in a statistically significant improvement in ocular function, macular function, cardiac function, brain growth and biochemistry, seizures, neuropathy and/or neurocognitive function. In other embodiments, the disclosed methods result in a statistically significant decrease in hydrocephalus. In further embodiments, the disclosed method result in an significant improvement in maculopathy, pigmentary retinopathy/retinal degeneration, and/or optic nerve atrophy.

In further embodiments, the method includes performing a) urine organic acid analysis; b) serum methylmalonic acid analysis; c) total plasma homocysteine analysis; d) plasma amino acid analysis; e) serum vitamin B12 level; and/or f) plasma acylcarnitine analysis. The disclosed methods result in a statistically significant improvement in one or more of these analyses.

### Pharmaceutical Compositions of Use in the Disclosed Methods

Pharmaceutical compositions are provided that are of use in any of the methods disclosed herein. These pharmaceutical compositions include a therapeutically effective amount of MeCbl and optionally a therapeutically effective amount OHCbl. In some embodiments, these compositions can include betaine, folate and/or folinic acid. These compositions can be formulated for intravenous, intramuscular, or subcutaneous administration. The pharmaceutical compositions can also be formulated for oral administration. Pharmaceutical compositions that include MeCbl are disclosed, for example, in U.S. Published Patent Application No. 2008/00394228, and U.S. Pat. No. 8,609,630, which are incorporated by reference herein. The pharmaceutical composition can be formulated for oral, parenteral (e.g., intramuscular, intraperitoneal, intravenous, intracisternal injection, infusion, subcutaneous injection, or implant), by inhalation spray, intranasal, transbuccal, mucosal, pulmonary, transdermal, liposomal, vaginal, rectal, or sublingual administration. The pharmaceutical compositions include suitable dosage unit formulations containing conventional non-toxic pharmaceutically acceptable carriers, adjuvants and vehicles appropriate for each route of administration.

In some embodiments, the pharmaceutical composition is formulated for intravenous, intramuscular, or subcutaneous administration. In other embodiments the pharmaceutical composition is formulated for oral or intranasal administration.

The pharmaceutical composition includes a therapeutically effective amount of MeCbl. In some embodiments, the pharmaceutical composition can include about 5 mg to about 10 grams of MeCbl. The pharmaceutical composition can include about 10 mg to about 10 grams of MeCbl, or about 5 mg to about 5 grams of MeCbl. The pharmaceutical composition can include any dose within these ranges.

In some embodiments, pharmaceutical composition can include about 5 mg to about 50 mg of MeCbl, such as about 10 to about 40 mg of MeCbl. The pharmaceutical composition can include about 5 to about 10, 15, 20, 25, 30, 35, 40, 45 or 50 mg of MeCbl. The pharmaceutical composition can include about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg of MeCbl.

In other embodiments, the pharmaceutical composition can include about 1 gram to about 8 grams, about 2 to about 10 grams, about 2 to about 5 grams, or about 5 to about 10 grams of MeCbl. The pharmaceutical composition can include about 1, 2, 3, 4, 5, 6, 7, 8, 9, or 10 grams of MeCbl.

In some embodiments, the pharmaceutical composition also includes a therapeutically effective amount of OHCbl. In non-limiting example, the ratio of MeCbl to OHCbl can be about 5:1, 4:1, 3:1, 2:1, or 1:1.

In some embodiments, the pharmaceutical composition can include about 5 mg to about 10 grams of OHCbl. The pharmaceutical composition can include about 10 mg to about 10 grams of OHCbl, or about 5 mg to about 5 grams of OHCbl. The pharmaceutical composition can include any dose within these ranges.

In some embodiments, pharmaceutical composition can include about 5 mg to about 50 mg of OHCbl, such as about 10 to about 40 mg of OHCbl. The pharmaceutical composition can include about 5 to about 10, 15, 20, 25, 30, 35, 40, 45 or 50 mg of OHCbl. The pharmaceutical composition can include about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg of OHCbl.

In other embodiments, the pharmaceutical composition can include about 1 gram to about 8 grams, about 2 to about 10 grams, about 2 to about 5 grams, or about 5 to about 10 grams of MeCbl. The pharmaceutical composition can include about 1, 2, 3, 4, 5, 6, 7, 8, 9, or 10 grams of MeCbl.

In further embodiments, additional active agents can also be included, such as, but not limited to, betaine, folate and/or folinic acid. Non-limiting amounts of betaine are up to about 15 grams (for example, about 25 mg/kg for a 70 kg human) Non-limiting amounts of folinic acid are about 350 mg to about 2.1 grams (about 5 to about 30 mg for a 70 kg human) Non-limiting amounts of folate are about 70 mg to about 700 mg (about 1 to about 10 mg/kg for a 70 kg human).

MeCbl, OHCbl, betaine, folate and folinic acid can be obtained from commercial suppliers (e.g., Sigma-Aldrich Fine Chemicals) or synthesized using methods known in the art. The composition can be formulated for administration by any route, including, but not limited to, oral, injection (such as subcutaneous, intramuscular, intradermal, intraperitoneal, and intravenous), sublingual, intrathecal, subretinal, intravitreous, rectal, transdermal (for example, topical), intranasal, vaginal, and inhalation. In a non-limiting example, the pharmaceutical composition is formulated for intramuscular administration. In another non-limiting example, the pharmaceutical composition is formulated for intravenous administration. In yet another non-limiting example, the pharmaceutical composition is formulated for oral administration.

An excipient can be selected to improve or enhance the solubility and/or stability of MeCbl and OHCbl. Substantial solubilization can be essentially complete solubilization (e.g., more than 80%, 90%, 95%, 96%, 97%, 98%, 99%). An “excipient” can be, or can include, at least one alcohol, which can be monohydric (i.e., an alcohol containing a single hydroxyl (—OH) group); dihydric (i.e., an alcohol containing two hydroxyl groups); trihydric (i.e., an alcohol containing three hydroxyl groups); or polyhydric (“polyols” contain three or more hydroxyl groups). The alcohol can be aliphatic (e.g., a paraffinic alcohol, such as ethanol, or olefinic, such as an allyl alcohol); alicyclic (e.g., a cyclohexanol); aromatic (such as phenol and benzyl alcohol); heterocyclic (e.g., furfuryl alcohol); or polycyclic (e.g., a sterol). Dihydric alcohols include glycols and derivatives thereof (diols), and trihydric alcohols include glycerols and derivatives thereof. More specifically, the excipients can be ethanol, propylene glycol (PG), polyethylene glycol (PEG (e.g., PEG 200 or PEG 300)), glycerol, mannitol, sorbitol, Tween 20, or a combination thereof. Other excipients, such as dimethylsulfoxide (DMSO), can also be used alone or in combination with one or more different excipients. An excipient can be, or can include, at least one salt former, including organic bases. Suitable organic bases include without limitation arginine, choline, choline chloride, L-lysine, D-lysine, ornithine, glucamine and its N-mono- or N,N-disubstituted derivatives, benethamine, banzathine, betaine, deanol, diethylamine, 2-(diethylamino)-ethanol, hydrabamine, 4-(2-hydroxyethyl)-morpholine, 1-(2-hydroxyethyl)-pyrrolidine, tromethamine, methylamine, diethanolamine, ethanolamine, ethylenediamine, 1H-imidazole, piperazine, triethanolamine (2,2′,2″-nitrilotris(ethanol), N-methylmorpholine, N-ethylmorpholine, pyridine, dialkylanilines, diisopropylcyclohexylamine, tertiary amines (e.g. triethylamine, trimethylamine) diisopropylethylamine, dicyclohexylamine, N-methyl-D-glutamine, 4-pyrrolidinopyridine, dimethylaminopyridine (DMAP), piperidine, isopropylamine, and meglumine. The excipient may be selected to ensure maximum activity and bioavailability of MeCbl and/or OHCbl without increasing any side effects. Compositions include liquid compositions (e.g. solutions, syrups, colloids, or emulsions).

The vitamin B₁₂-containing compositions can be solutions that include an excipient, which can be, or can include, at least one monohydric, dihydric, trihydric, or polyhydric alcohol which can be aliphatic, alicyclic, aromatic, or polycyclic. More specifically, the excipients can be ethanol, propylene glycol, polyethylene glycol (PEG (e.g., PEG 200 or PEG 300)), glycerol, mannitol, sorbitol, TWEEN® 20, dimethylsulfoxide (DMSO), or a combination thereof.

In some embodiments, the excipient is a polyethylene glycol (PEG), in particular PEG 200 or PEG 300, at least 15%, 20%, 30% or 40% ethanol, or propylene glycol, or combinations thereof. In particular aspects the excipient is a combination of propylene glycol and ethanol, more particularly 10-60%, 10-40%, or 20-40% propylene glycol and 5-20% ethanol, most particularly 20-40% propylene glycol and 10%, 15%, or 20% ethanol.

In other embodiments, the pharmaceutical composition includes a salt former, more particularly an organic base, most particularly choline or choline chloride. The molar ratio of a salt former, in particular choline or choline chloride, to MeCbl and/or OHCbl may be about 1:1 to about 1:15 or 1:1 to about 1:10, more particularly about 1:1, 1:3, 1:5 or 1:10. In some embodiments the amount of salt former, in particular choline or choline chloride, in a composition of the invention is about 5-100 mg/ml, about 5-70 mg/ml, about 5-50 mg/ml, about 5-25 mg/ml, or about 5-20 mg/ml.

The pharmaceutical composition of use in the disclosed methods can also comprise suitable pharmaceutical carriers, vehicles, or diluents selected based on the intended form of administration, and consistent with conventional pharmaceutical practices. Suitable pharmaceutical carriers, vehicles, or diluents are described in the standard text, Remington's Pharmaceutical Sciences (Mack Publishing Company, Easton, Pa., USA 1985). By way of example, suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, targeting agents, and coloring agents may also be combined in the compositions or components thereof. Compositions can be formulated as neutral or pharmaceutically acceptable salt forms.

The pharmaceutical composition can include a unit dosage of at least MeCbl and at least one excipient to provide beneficial effects. A “unit dosage” refers to a unitary i.e. a single dose which is capable of being administered to a human subject, and which may be readily handled and packed, remaining as a physically and chemically stable unit dose comprising either the active agents as such or a mixture with one or more excipients. The single dose can optionally include OHCbl.

The pharmaceutical composition can be sterilized by, for example, by filtration through a bacteria-retaining filter, addition of sterilizing agents to the composition, irradiation of the composition, or heating the composition. Alternatively, the compounds or compositions can be provided as sterile solid preparations e.g. lyophilized powder, which are readily dissolved in sterile solvent immediately prior to use.

Intranasal compositions for the administration of MeCbl are disclosed, for example, in U.S. Published Patent Application No. 2020/0009179, incorporated herein by reference. In some embodiments, these compositions include at least one gelling agent and a permeation enhancer. The permeation enhancer can be lecithin and/or di-ethylene glycol monoethyl ether. In some embodiments, the permeation enhancer can include chitosan, poloxamer, heptyl glucoside, polyoxyethylene sorbitan monolaurate (TWEEN® 20, TWEEN® 60, TWEEN® 80), dimethyl isosorbide, caprylocaproyl polyoxyl-8 glycerides, cyclodextrin, cyclic urea and amino acids. Amino acids such as glycine, cysteine, leucine, isoleucine, alpha-amino butyric acid, or the like can be used as permeation enhancer. In some embodiments, the gelling agent includes gellan gum, xyloglucan, alginic acid, sucrose cross-linked pectin, poloxamers or the like or the mixture thereof. The pharmaceutical compositions can also include a thickening agent, such as naturally-occurring polymeric materials for example, but not limited to, locust bean gum, sodium alginate, sodium caseinate, egg albumin, gelatin agar, carrageenin gum, quince seed extract, starch, chemically modified starches and the like; semi-synthetic polymeric materials such as cellulose ethers (e.g. hydroxyethyl cellulose, methyl cellulose, carboxymethyl cellulose, hydroxy propylmethyl cellulose), polyvinylpyrrolidone, polyvinylalcohol, guar gum, hydroxypropyl guar gum, soluble starch, cationic celluloses, cationic guars and the like, or synthetic polymeric materials such as carboxyvinyl polymers, polyvinylpyrrolidone, polyvinyl alcohol polyacrylic acid polymers, polymethacrylic acid polymers, polyvinyl acetate polymers, polyvinyl chloride polymers, polyvinylidene chloride polymers and the like, and hyaluronic acid.

In some embodiments, the pharmaceutical composition include a humectant, such as cetyl palmitate, glycerol (glycerin), PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ benzoates, dimethiconol, ethylene glycol, propylene glycol, hexylene glycol, theobroma grandiflorum seed butter, ceramides (for example, ceramide 2 or ceramide 3), hydroxypropyl bispalmitamide MEA, hydroxypropyl bislauramide MEA, hydroxypropyl bisisostearamide MEA, 1,3-bis(N-2-(hydroxyethyl)stearoylamino)-2-hydroxy propane, bis-hydroxyethyl tocopherylsuccinoylamido hydroxypropane, urea, aloe, allantoin, glycyrrhetinic acid, and dicaprylate/dicaprate. In some embodiments, humectant is used in the range of about 1 to 5% w/v. In an embodiment, the humectant is glycerin, such as in an amount of about 2% w/v.

In further embodiment, the pharmaceutical composition includes a preservative. Suitable preservatives include, but are not limited to, benzalkonium chloride, methyl, ethyl, propyl or butylparaben, benzyl alcohol, phenylethyl alcohol, benzethonium, chlorobutanol, potassium sorbate or combination thereof. In some embodiments, the preservative is benzyl alcohol, phenyl ethyl alcohol or their mixture.

In more embodiments, the pharmaceutical composition includes a flavoring agent. The flavoring agent can be chosen from natural and synthetic flavoring liquids such as volatile oils, synthetic flavor oils, flavoring aromatic and oils, liquids, oleoresins or extracts derived from plants, leaves, flowers, fruits, stems and combinations thereof. Non-limiting representative examples of volatile oils include spearmint oil (Novamint Spearmint), cinnamon oil, oil of wintergreen (methyl salicylate), peppermint oil, menthol, lavender, lotus, rose, saffron, jasmine, eugenol, clove oil, bay oil, anise oil, eucalyptus oil, thyme oil, cedar leaf oil, oil of nutmeg, allspice oil, oil of sage, mace extract, oil of bitter almond, and cassia oil. Various artificial, natural or synthetic flavors can also be used including fruit flavors such as vanilla, and citrus oils including lemon, orange, grape, lime and grapefruit and fruit essences including apple, pear, peach, grape, strawberry, raspberry, cherry, plum, pineapple, apricot and so forth. Other useful flavoring agents include aldehydes and esters such as benzaldehyde (cherry, almond), citral, i.e., alphocitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenal (citrus, mandarin), or the like or the mixtures thereof.

Vehicles of use in these pharmaceutical compositions include, but are not limited to, saline, water, dextrose or combinations thereof. In some embodiments, the vehicle is water. The amount of vehicle depends on the amounts of the other ingredients present in the composition. The amount of vehicle is a sufficient amount (q.s.) that is required to establish a specified volume.

The pharmaceutical composition can be formulated for oral administration, see for example, U.S. Pat. No. 8,609,630, incorporated herein by reference. In some embodiment, these formulations include the MeCbl and optionally OHCbl, a carrier suitable for forming a solid or semi-solid carrier matrix. In some embodiments, the carrier is a sugar, sugar alcohol, PEG, starch, gum, polymer, or combination thereof. In some embodiments, the carrier comprises isomalt, a PEG, or a combination thereof. In some embodiments, the PEG is PEG-8000. In some embodiments, the carrier comprises PEG-8000, and isomalt, or a derivative thereof.

In more embodiments, the pharmaceutical compositions further comprise a lubricant. In some embodiments, the lubricant is magnesium stearate. In some embodiments, the compositions further comprise a flavoring agent. In some embodiments, the flavoring agent is: apple, almond, amaretto, anise, apricot, banana, banana orange, blackberry, black cherry, black currant, black walnut, blueberry, brandy, bubblegum, butter rum, butterscotch, caramel, cinnamon, citrus, citrus punch, cherry, chocolate, chocolate banana pie, chocolate covered cherry, chocolate hazelnut, cloves, coconut, coffee, cotton candy, creme de menthe, egg nog, English toffee, ginger, grape, grapeade, grape bubblegum, grapefruit, fig, hazelnut, honey, Irish cream, kiwi, lavender, lemon, licorice, lime, maple, marshmallow, mint, mocha, molasses, orange, orange cream, passion fruit, peach, pecan, peppermint, pina colada, pineapple, pistachio, plum, praline, pomegranate, pumpkin, raspberry, red licorice, root beer, sassafras, sour apple, spearmint, strawberry, strawberry cream, tangerine, tropical fruit, tutti-fruiti, vanilla, walnut, watermelon, white chocolate, wild cherry, or wintergreen. In some embodiments, the flavoring agent is cherry.

In some embodiments, the composition is formulated as a lozenge, a candy, a wafer, a tablet, a patch, a film, a spray, a lip balm, or gum. In some embodiments, the composition is formulated as a lozenge. In some embodiments, the MeCbl, is includes as about 0.5-5% weight/weight (w/w) of the composition, such as from about 1% to 5% w/w. In some embodiments, the compositions comprise isomalt, polyethylene glycol, flavoring, and magnesium stearate.

In some embodiments, the pharmaceutical compositions further comprise antimicrobial agents, plasticizing agents, sulfur precipitating agents, saliva stimulating agents, cooling agents, surfactants, stabilizing agents, emulsifying agents, thickening agents, binding agents, coloring agents, sweeteners, fragrances, and the like. Exemplarily sulfur precipitating agents useful in the present invention include metal salts such as copper salts (such as copper gluconate) and zinc salts (such as zinc citrate and zinc gluconate). Exemplarily saliva stimulating agents include, but are not limited to, food acids such as citric, lactic, malic, succinic, ascorbic, adipic, fumaric and tartaric acids.

Kits are also of use in the disclosed methods. These kits include a pharmaceutical composition comprising MeCbl, and instructions for use. In some embodiments, the pharmaceutical composition further comprises a carrier. In some embodiments, the pharmaceutical composition also includes OHCbl. In certain embodiments, the kit includes a pharmaceutical composition disclosed herein and instructions for storage, administration, dosing, and information on treatment of a human subject with a cblC deficiency, or the treatment of a female human subject pregnant with a fetus with a cblC deficiency. In yet another embodiment is an article of manufacture comprising a composition or formulation disclosed herein and an apparatus to dispense or administer the formulation to a given patient, such as a container for housing the composition, or a device for administration. In some embodiments, the kit includes the composition described herein, and instructions for using the kit.

In further embodiments, the kits include packaging materials including, but not limited to, blister packs, bottles, tubes, inhalers, pumps, bags, vials, containers, bottles, and any packaging material suitable for a selected formulation and intended mode of administration and treatment, including labels listing contents and/or instructions for use, and package inserts, with instructions for use. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

In certain embodiments, the composition is presented in a pack or dispenser device which contains one or more unit dosage forms. In another embodiment, the pack for example contains metal or plastic foil, such as a blister pack. In a further embodiment, the pack or dispenser device is accompanied by instructions for administration. In yet a further embodiment, the pack or dispenser is also accompanied with a notice associated with the container in form prescribed by a governmental agency regulating the manufacture, use, or sale of pharmaceuticals, which notice is reflective of approval by the agency of the form of the drug for human administration. In another embodiment, such notice, for example, is the labeling approved by the U.S. Food and Drug Administration for prescription drugs, or the approved product insert.

The disclosure is illustrated by the following non-limiting Examples.

### EXAMPLES

Disclosed below is the development of the a viable Mmachc mouse model which recapitulates several hallmarks of a cblC deficiency, including growth failure, brain pathology and biochemical perturbations. The effect of OHCbl treatment on survival was confirmed, and MeCbl was shown to be therapeutic. Treatment was also achieved with AAV gene transfer, where a single neonatal injection of an AAV vector, expressing either the mouse or human MMACHC gene, provided long-term metabolic control and produced equivalent survival compared to chronic, injectable OHCbl treatment in the mutant cblC mice.

### Example 1

### Materials and Methods

Generation of Mmachc mouse model: TALENs were designed to target exon 2 the following sequence in Mmachc 5′-TACCCTGGCCTTCCTGGTACTCAGCACACCTGCTATGTTTGACAGAGCCCTCA-3′ (SEQ ID NO: 1) with the putative cleavage site underlined. RNA was injected into embryos with FVB/N background and then crossed to C57B6 mice. Variants in Mmachc are described using the following transcript: GENBANK® Accession No. NM_025962.3, as available on Oct. 1, 2020, incorporated herein by reference.

Genotyping and breeding scheme: Tail biopsies were performed routinely at 10-15 days of life or in rare cases in the first day of life and DNA was extracted using the Qiagen DNEASY® Spin Kit or the MYTAQ® Extract-PCR Kit. Mmachc genotype was determined by a fluorescent PCR method described previously²² or by Droplet Digital PCR (ddPCR) Mutation Detection Assay (Bio-Rad). ddPCR genotyping was performed by mixing 2 ul of 1:10 diluted DNA, 1.1 ul probe-primer mix, and 7.9 ul water. The primer-probe mix contained the wild-type/mutant primer and fluorescent FAM and HEX probes targeting the mutant and wild-type alleles, respectively. Sample droplets were generated by the QX200 Droplet Digital PCR system, PCR amplified in a thermocycler, and DNA quantified by droplet fluorescence. Mmachc homozygous mutant mice and their heterozygote or wild type control littermates were generated from heterozygous parental crosses unless otherwise noted. In order to eliminate the Pdeb(rdl) mutation present in the FVB/N genetic background which causes retinal degeneration, Mmachc carrier mice were crossed to C57BL/6J mice. Subsequent generations of Mmachc carriers were genotyped for the Pdeb(rdl) mutation using a published PCR method (Gimenez and Montouliu Lab Anim 35, 153-6 (2001)), and mice homozygous for the functional Pdeb(rdl) allele were selected for further breeding. Mice were fed regular or high fat chow and weaned at P21-28.

Survival and phenotype in Mmachc mice: Mendelian ratios at birth, survival, and relative weight: To determine survival, litters were assessed daily after birth and dead pups were noted, removed, and genotyped when tissue was available. Age at time of death was recorded. Weights were measured monthly and the weight of mutants relative to average weight of control littermates was calculated.

Pathology: Tissues from three Mmachc^{de13/de13} mutants and three control littermates at 1 month of age were studied by H&E staining

Metabolite measurements: Blood samples were collected in heparinized capillary tubes and centrifuged at 2000 rpm for 10 min at 4C. Plasma was diluted 1:40 in water for methylmalonic acid and methylcitrate measurements and undiluted plasma was used for homocysteine and cystathionine measurements. Plasma concentrations were determined by gas chromatography-mass spectrometry (GC-MS).

Mouse Embryonic Fibroblasts: Female mice were bred and checked daily for plugs. The morning a plug was found was designated at P0.5. Pregnant dams were sacrificed, and embryos were collected at E12.5. Tissue was saved for genotyping and fibroblast cell culture.

Cobalamin Treatment: OHCbl and MeCbl were obtained from College Pharmacy (Colorado Spring, CO) or prepared at 25 mg/ml concentration and subsequently diluted with 0.9% saline solution to a working solution of 5 mg/ml. OHCbl or MeCbl was delivered by intraperitoneal injections for pups ages P0-P14 and subcutaneous injections for mice post weaning. For in utero treatment studies, pregnant dams were treated with 1 mg/week (100 mg/kg) via subcutaneous injections throughout pregnancy and weaning. For chronic therapy 50 μg/kg dosing was used.

AAV Vector Design, Production, and Delivery: Two adeno associated viral vectors were designed: AAVrh10-Mmachc, containing the murine Mmachc coding sequence packaged into a rh10 serotype capsid, and AAV9-_{co}MMACHC, containing a codon optimized human MMACHC coding sequence packaged into an AAV9 serotype capsid. AAVs were produced as described previously. AAV viral vectors were delivered via intrahepatic injection at a dose of 1×10¹¹ GC per pup in the neonatal period (P0-2). ddPCR was conducted using a CMV/CBA probe spanning the promoter/enhancer, reference albumin Dissection of Pregnant Dams: Pregnant dams were either untreated, treated with OHCbl, MeCbl or the combination (1 mg per week) during pregnancy as described and sacrificed at E18.5. Embryos were photographed, tails were collected for genotyping, and the remaining tissue was collected and flash frozen. Embryo weights were measured and the weight of mutants relative to average weight of control littermates was calculated. Crown to rump length of each embryo was also determined using ImageJ software.

Statistics: Graphpad Prism Software was used for statistical analysis. All data are presented as mean±SD and significance is indicated as follows: p<0.05(*), p<0.01(**), p<0.001 (***), and p<0.0001(****). Kaplan-Meier survival curve significance was determined using the Log-Rank Mantel-Cox test, genotype distribution significance was assessed using the chi-square test, and all other statistics were calculated using the unpaired t-test.

### Example 2

Mmachc^{-/-} Mice Display Neonatal Lethality Reduced Survival, and Metabolic Perturbations

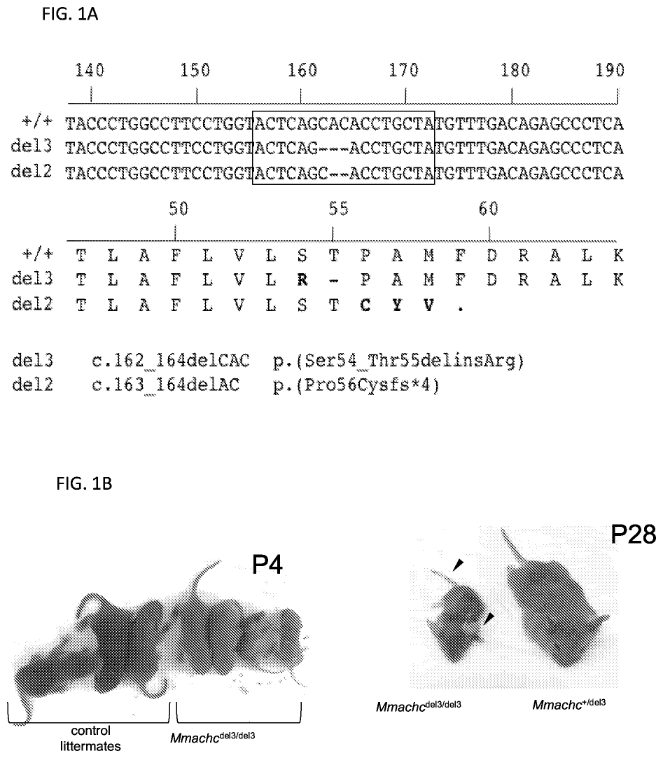

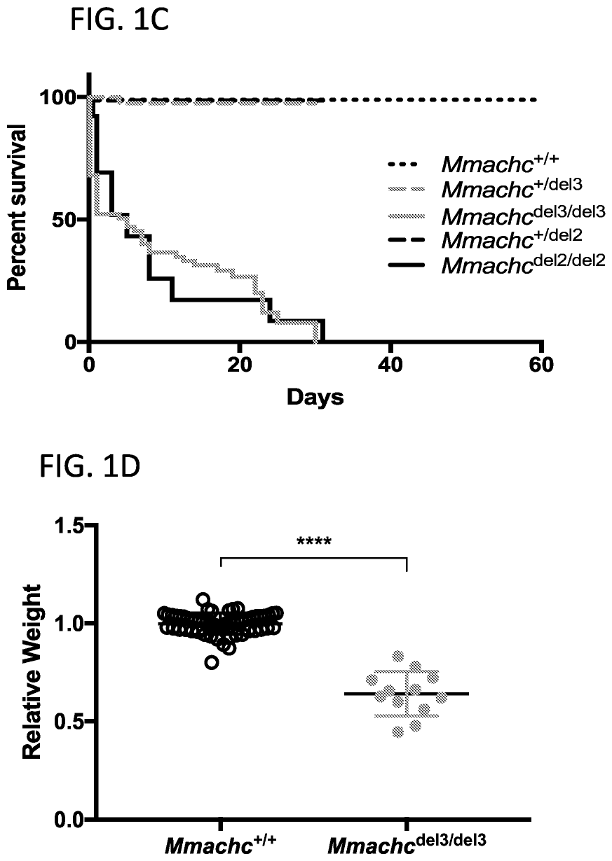

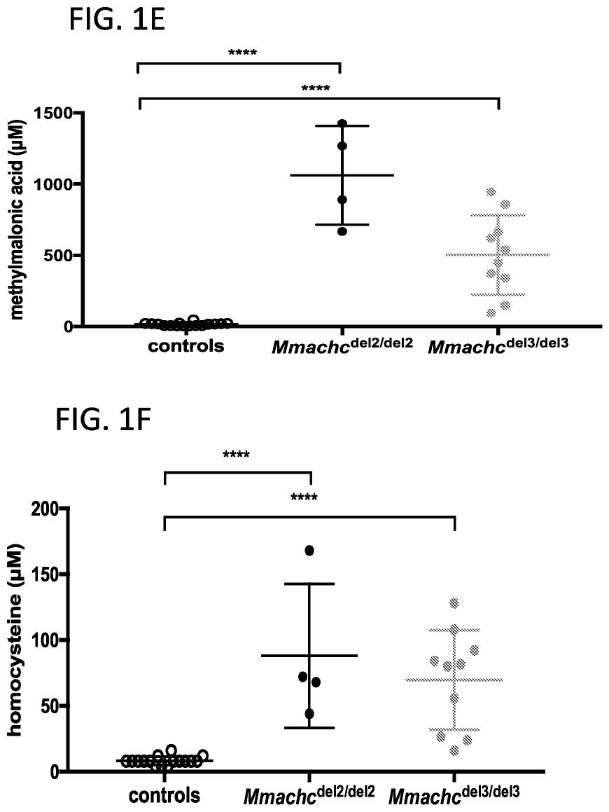

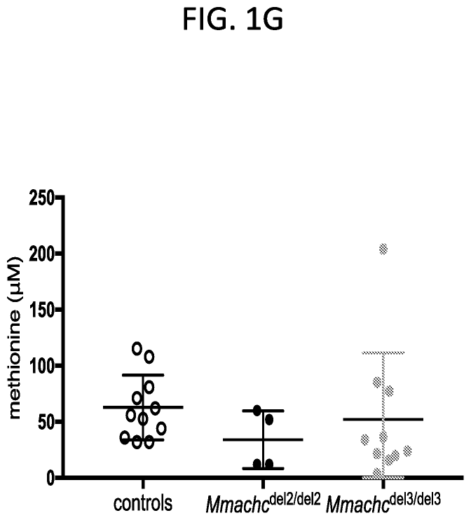

To create a mouse model of cblC, TALENs were synthesized to target exon 2 of Mmachc, to attempt to model the common frameshift variant in humans, c.271dupA p.Arg91Lysfs* 14. Fluorescent PCR capillary electrophoresis and Sanger sequencing was used to screen founder mice and ten alleles were recovered. Two alleles were selected for in depth characterization: c.165_166de1AC p.(Pro56Cysfs*4) and will be referred to as Mmachc^{de12} and c.162_164de1CAC p.(Ser54_Thr55delinsArg) as Mmachc^{de13} below (FIG. 1A). The de12 allele is a frameshift variant predicted to result in no functional Mmachc protein and the deli allele is predicted to result in an in-frame indel.

In the first week of life Mmachc^{de13/de13} mice were readily identifiable due their small size and delayed fur growth and hypopigmentation of their ears and tail (FIG. 1B). The survival of the Mmachc^{-/-} mice was drastically reduced compared with controls (p<0.0001), with a median survival of 5 days and no mutants surviving beyond 31 days (FIG. 1C). Surviving mutants displayed significant growth impairment; by 2 weeks they were 35% smaller than wildtype and heterozygous littermates (FIG. 1D). cblC mutant mice were extremely difficult to generate disturbed Mendelian ratios were observed, with a reduction in the number of mutant pups for both alleles at birth: Mmachc^{de12} N=134, 19 litters, p<0.001 and Mmachc^{de13} N=771, 127 litters, p<0.0001 (Table 1).

TABLE 1
Genotype distribution of Mmachc^{-/-} mice at postnatal day 0-2

| | | genotype distribution | | | | | | | |
|---|---|---|---|---|---|---|---|---|---|
| | | mice at E18.5 | | | | | | | |
| | | n (%) | | | | | | | |
| mouse line | treatment | +/+ | +/− | −/− | total mice | # litters | χ² | df | p-value |
| Mmachc^{del2} | none | 27 | 41 | 19 | 87 | 12 | 1.759 | 2 | 0.4151 |
| | | (31) | (47) | (22) | | | | | |
| Mmachc^{del3} | none | 14 | 45 | 14 | 73 | 9 | 3.959 | 2 | 0.1381 |
| | | (19) | (62) | (19) | | | | | |
| Mmachc^{del3} | prenatal | 14 | 28 | 19 | 61 | 6 | 1.230 | 2 | 0.5408 |
| | OHCbl | (23) | (46) | (31) | | | | | |

TABLE 2
Genotype distribution of Mmachc^{-/-} mice at embryonic day E18.5

| | | genotype distribution | | | | | | | |
|---|---|---|---|---|---|---|---|---|---|
| | | mice at birth | | | | | | | |
| | | n (%) | | | total | # | | | |
| mouse line | treatment | +/+ | +/− | −/− | mice | litters | χ² | df | p-value |
| Mmachc^{del2} | none | 39 (29) | 81 (61) | 14 (10) | 134 | 19 | 15.179 | 2 | 0.0005 |
| Mmachc^{del2} | prenatal | 76 (29) | 171 (66) | 13 (5) | 260 | 41 | 56.392 | 2 | <0.0001 |
| | OHCbl | | | | | | | | |
| Mmachc^{del2} | prenatal | 14 (18) | 41 (53) | 22 (29) | 77 | 12 | 1.987 | 2 | 0.3703 |
| | MeCbl | | | | | | | | |
| Mmachc^{del2} | prenatal | 12 (25) | 24 (50) | 12 (25) | 48 | 6 | 0 | 2 | 1.00 |
| | OHCbl + | | | | | | | | |
| | MeCbl | | | | | | | | |
| Mmachc^{del3} | none | 225 (29) | 453 (59) | 93 (12) | 771 | 127 | 68.837 | 2 | <0.0001 |
| Mmachc^{del3} | prenatal | 105 (30) | 192 (54) | 57 (16) | 354 | 51 | 15.559 | 2 | 0.0004 |
| | OHCbl | | | | | | | | |
| Mmachc^{del3} | prenatal | 38 (31) | 76 (63) | 7 (6) | 121 | 20 | 23.826 | 2 | <0.0001 |
| | MeCbl | | | | | | | | |
| Mmachc^{del23} | none | 32 (38) | 50 (60) | 2 (2) | 84 | 9 | 24.476 | 2 | <0.0001 |

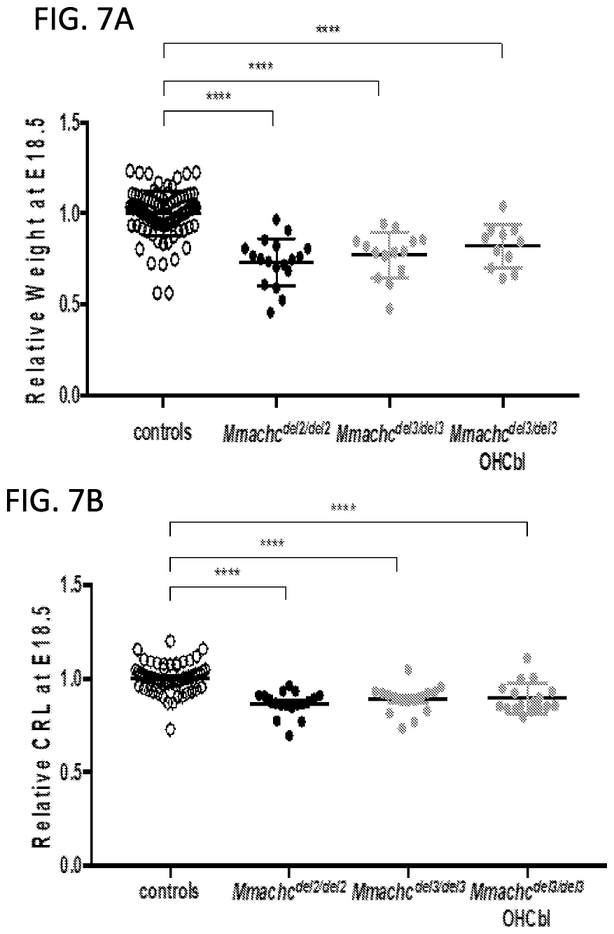

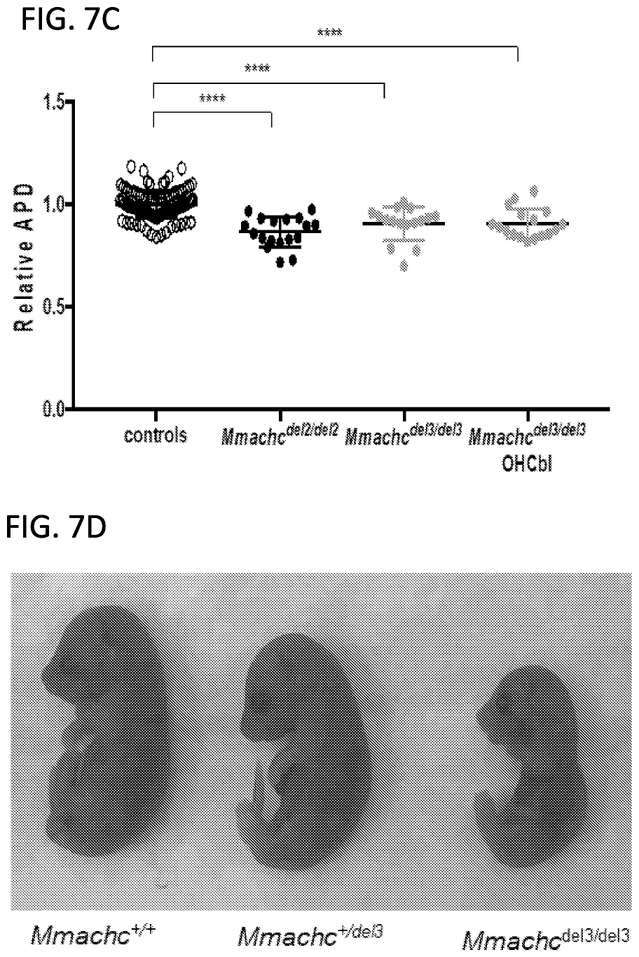

To investigate whether disturbed ratios were due to failure of embryonic development as reported in a previous cblC mouse model, dissections of pregnant dams were performed at E18.5 and displayed the expected number of mutants embryos (Table 2). However, mutant embryos weighed less and growth parameters (crown rump length and abdominal anterior-posterior diameter (APD)) were decreased compared to their littermates (p<0.0001) (FIG. 7A-D). Next, prenatal treatment was tried with OHCbl as has been described in humans (Trefz et al., Mol Genet Metab Rep 6, 55-9 (2016); Huemer et al., J Pediatr 147, 469-72 (2005)). Prenatal OHCbl supplementation did not improve embryonic growth parameters for Mmachc^{de13/de13} mutants and the disturbed genotype ratios persisted in both Mmachc^{de12} and Mmachc^{de13} prenatal OHCbl treated litters (Table 1).

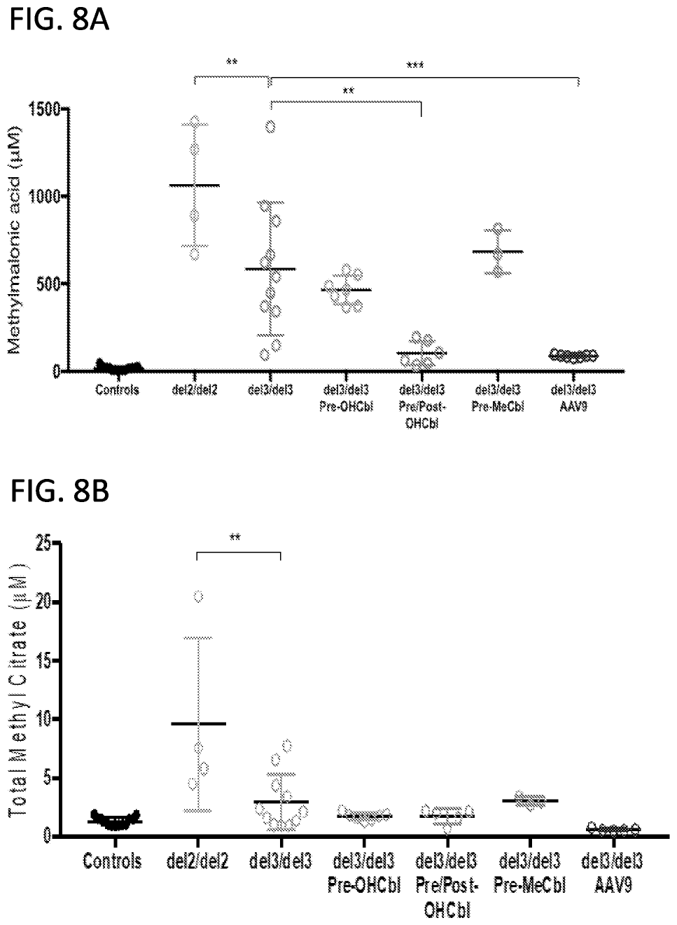

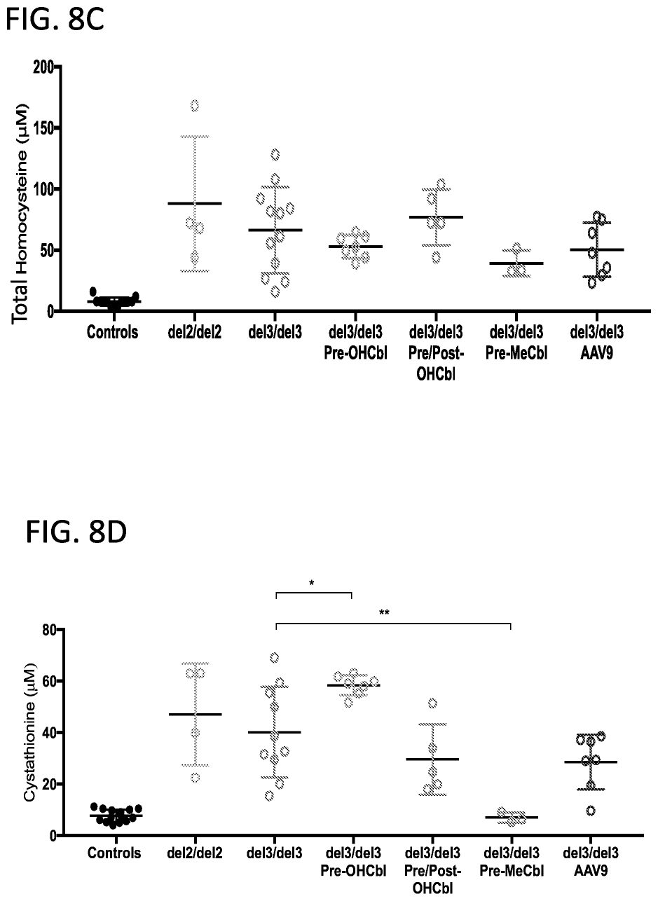

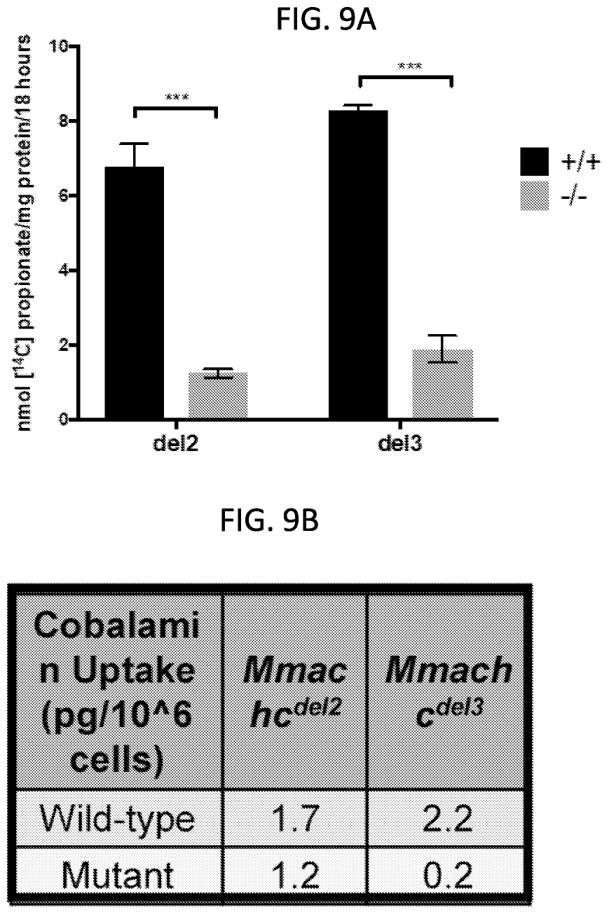

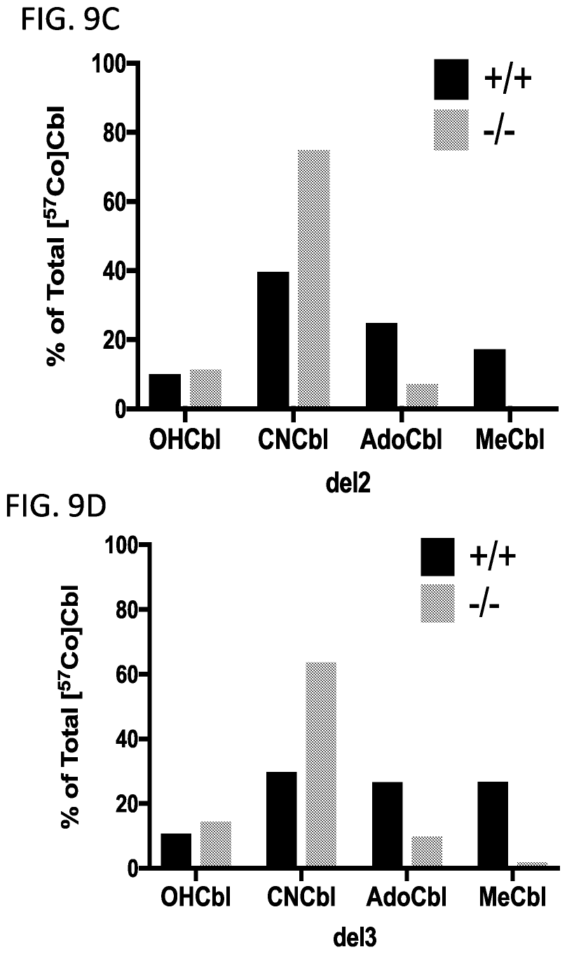

As expected, surviving Mmachc^{-/-} mice displayed metabolic perturbations similar to the patients affected with cblC: elevated plasma methylmalonic acid and homocysteine, and decreased methionine (FIG. 1E-G). Other related biochemical parameters cystathionine and total methylcitrate were also significantly elevated (FIG. 8). Both Mmachc^{de12/de12} and Mmachc^{de13/de13} mouse embryonic fibroblasts had decreased [¹³C]propionate incorporation into protein, decreased uptake of [⁵⁷Co]CNCbl and decreased synthesis of AdoCbl and MeCbl compared with fibroblasts derived from their wildtype littermates (FIG. 9). This is consistent with the characteristics of skin fibroblasts derived from individuals with cblC (Cooper and Rosenblatt, Annu Rev Nutr 7, 291-320 (1987)).

### Example 3

Survival and Metabolites Following OHCbl Treatment

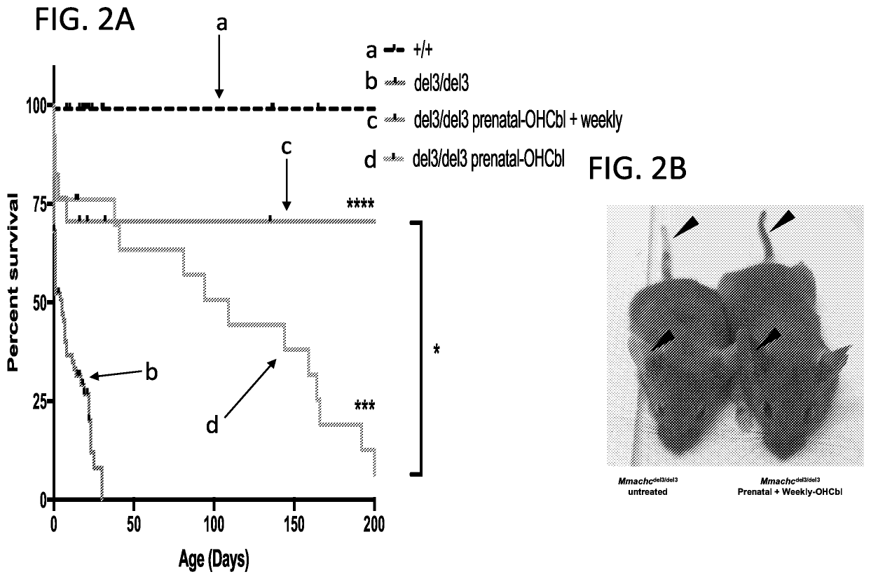

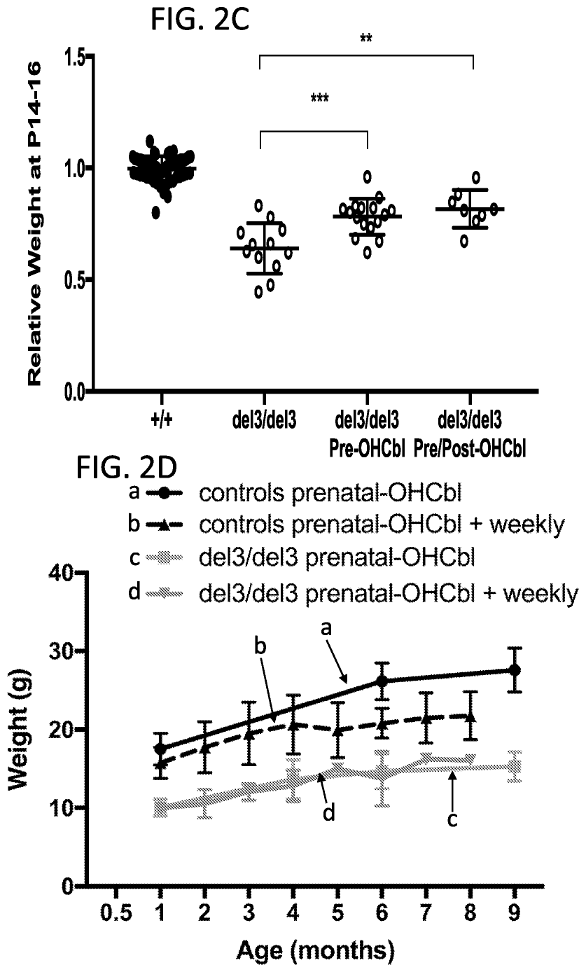

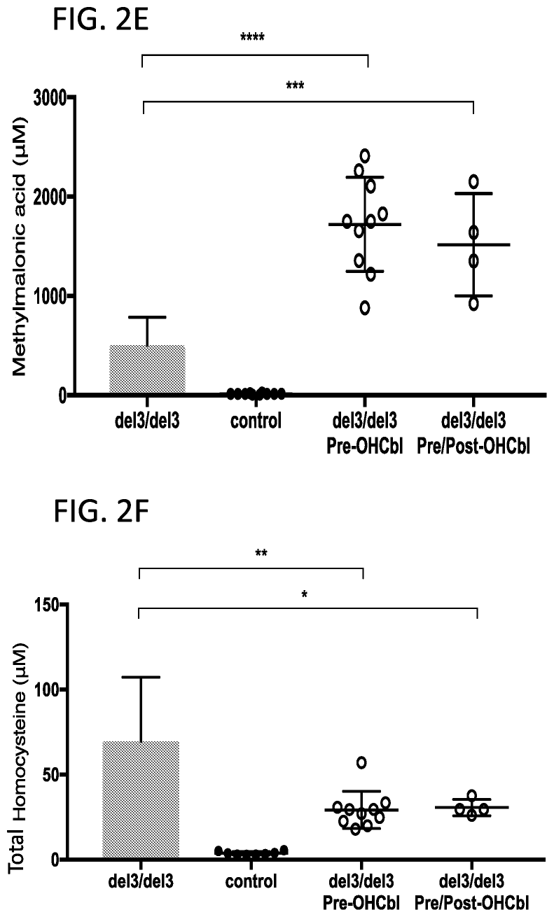

The long-term survival of Mmachc^{de13/de13} was examined following prenatal treatment with OHCbl only or prenatal treatment combined with chronic (weekly) treatment with OHCbl. Prenatal OHCbl alone significantly improved survival with median survival of 109 days compared to 5 days in the untreated mutants(p<0.001). With prenatal+weekly OHCbl, we observed long term survival with the longest surviving >1 year which was significantly improved from prenatal treatment alone (p<0.05) and untreated mice (p<0.0001) (FIG. 2A). Interestingly, decreased hypopigmentation was observed in the ears and tail of long term OHCbl treated mutants (FIG. 2B). Growth outcomes in both treated groups were similar. There was improvement in the weight of the mutant mice compared with untreated. However treated mutants remained growth impaired compared with their control littermates at 2 weeks (FIG. 2C) and later timepoints (FIG. 2D).

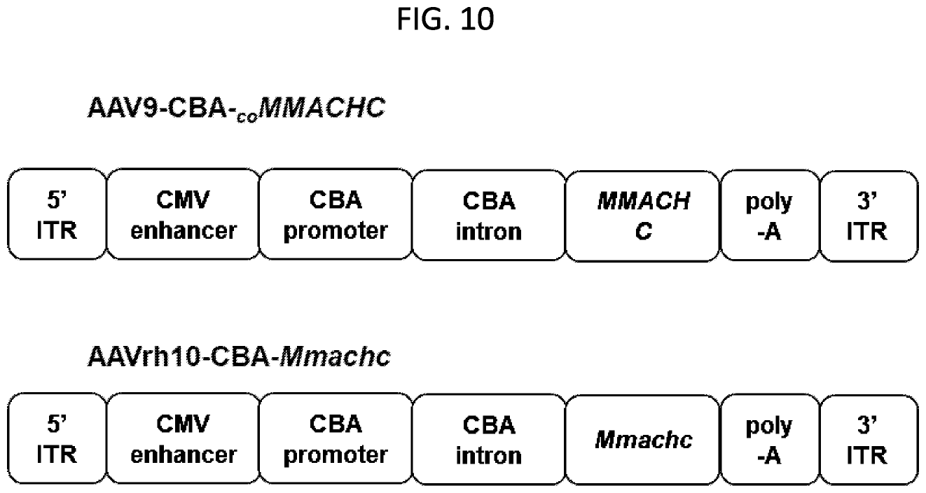

Biochemical parameters were also measured at two weeks and MMA levels were decreased with chronic OHCbl treatment but not surprisingly no decrease in MMA was observed with prenatal treatment only (FIG. 10A). Homocysteine remained elevated at untreated levels in both prenatal OHCbl treated mutants and prenatal/postnatal treated mice (FIG. 10C). Clinically, prenatal/postnatal OHCbl treated mice were often indistinguishable from controls as they did not display the hypopigmentation in tail and ears observed in untreated Mmachc^{de13/de13} mice (FIG. 2B). The adult Mmachc^{de13/de13} mutants remained growth impaired compared with their littermates (FIG. 2D) and even though they displayed clinical improvement and long-term survival, MMA and homocysteine remained elevated (FIGS. 2D, E).

### Example 4

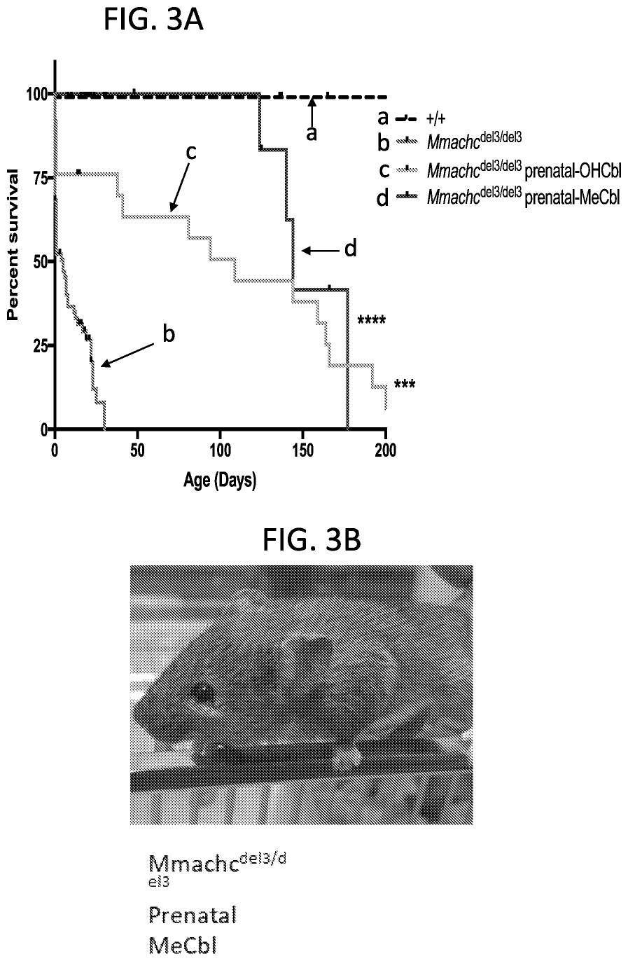

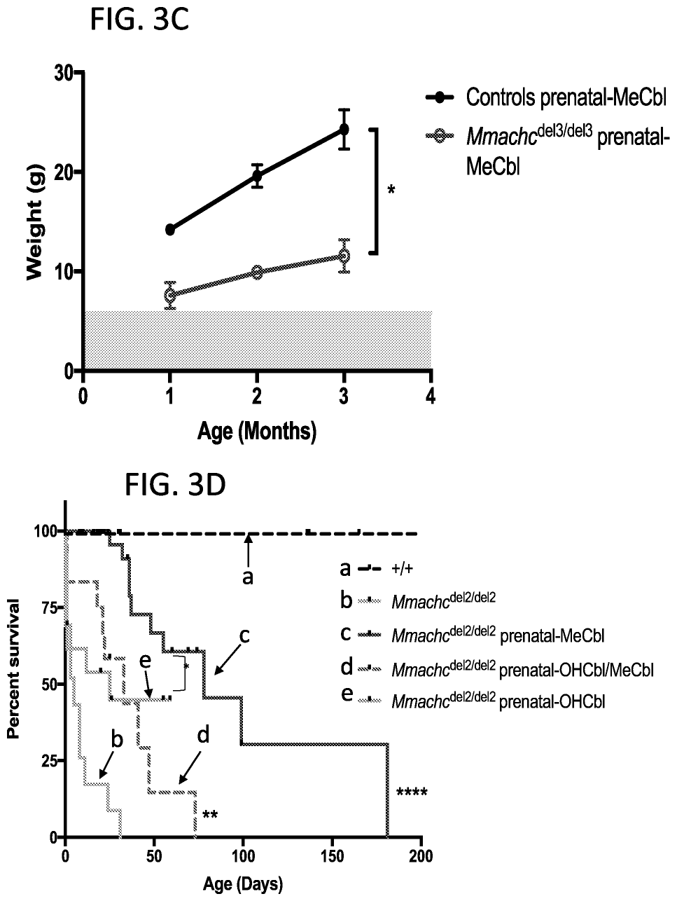

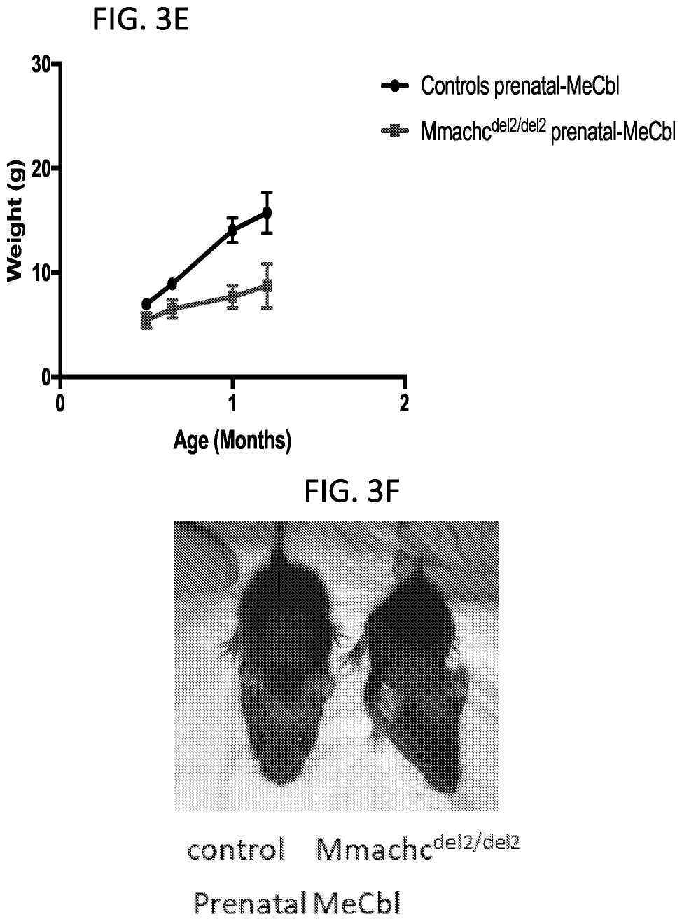

MeCbl treatment Despite documented MeCbl deficiency in cblC fibroblasts, there are only a few case reports about MeCbl treatment in patients with uncertain efficacy (Linnell et al., Journal of Inherited Metabolic Disease 6, 137-139 (1983); Ribes et al., Eur J Pediatr 149, 412-5 (1990); Smith eta;., Mol Genet Metab 88, 138-45 (2006)). Improved growth parameters were observed in a zebrafish model (Sloan et al., Hum Mol Genet 29, 2109-2123 (2020)) and these observations were extended to mice where Mmachc pregnant dams were treated with MeCbl. Similar to untreated and prenatal OHCbl treated dams, disturbed genotype ratios were observed in the Mmachc^{de13} model, with less mutants identified at P0-P2 (p<0.0001) (Table 1). Survival of the Mmachc^{de13/de13} mutants was improved (p<0.0001 vs untreated) with a striking improvement in early survival—100% of mutants were alive at 100 days vs 50% of the prenatally OHCbl treated mutants although median survival was not statistically different from prenatal OHCbl (OHCbl 109 days vs MeCbl 144 days, p=0.3369) (FIG. 3A). Despite improved survival, the prenatal MeCbl treated mutants had poor growth. They were 48% of the size of their littermates at 3 months (p<0.05, FIG. 3C). Interestingly the mice treated prenatally with MeCbl had minimal pigmentation differences of their fur, ears and tails making them difficult to distinguish from their littermates in the first weeks of life with the exception of speckled pigmentation of the ears as shown in FIG. 3B. This improvement in pigmentation is similar to what we observed in mice treated weekly with OHCbl (FIG. 2A).

In Mmachc^{de12} dams treated prenatally with MeCbl we unexpectedly observed restored genotypes 1:2:1 ratio at P0-P2 (p=0.3703). Similar effects were noted with combination MeCbl+OHCbl treatment (p=1.0) (Table 1). This was surprising given that the Mmachc^{de12/de12} mutants were extremely difficult to generate with prenatal OHCbl therapy (OHCb1:13 mutants in 41 litters vs MeCbl: 22 mutants in 12 litters, Table 1). Prenatal MeCbl also significantly improved survival compared with untreated (p<0.0001), prenatal OHCbl (p=0.0470) and combination treatment (p=0.0003) (FIG. 3D). Surprisingly 100% of Mmachc^{de12/de12} mutants survived beyond 30 days. The Mmachc^{de12/de12} mutants appeared growth impaired but this was not significant at 1 month (FIG. 3E). Similar improvement in pigmentation of the ears was observed with Mmachc^{de12/de12} mutants treated prenatally with MeCbl (FIG. 3F).

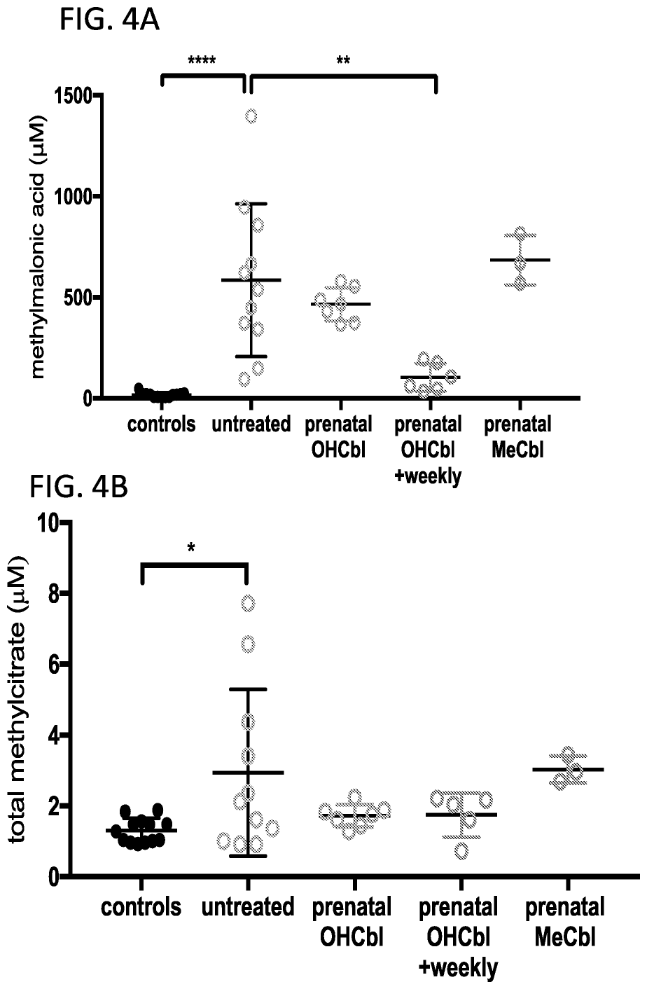

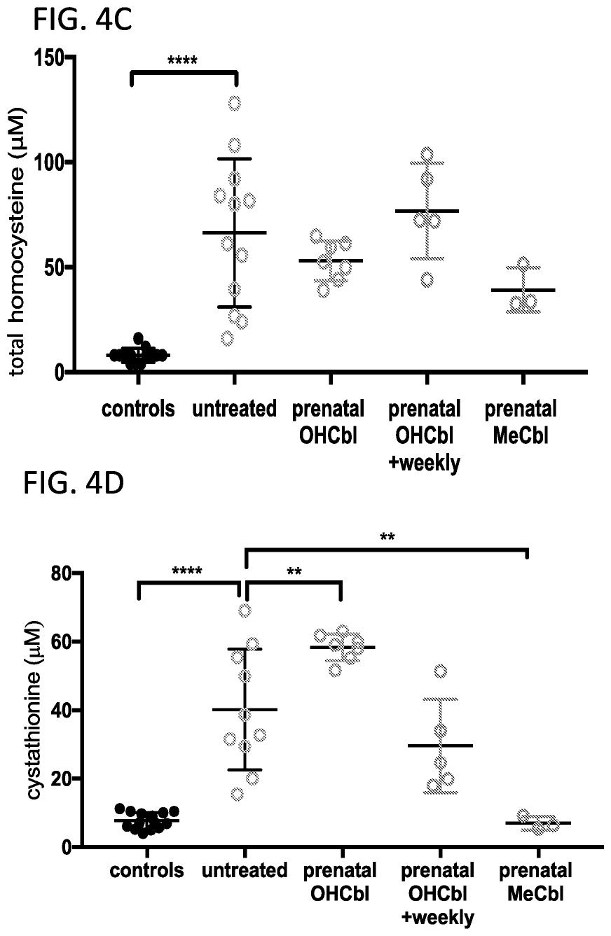

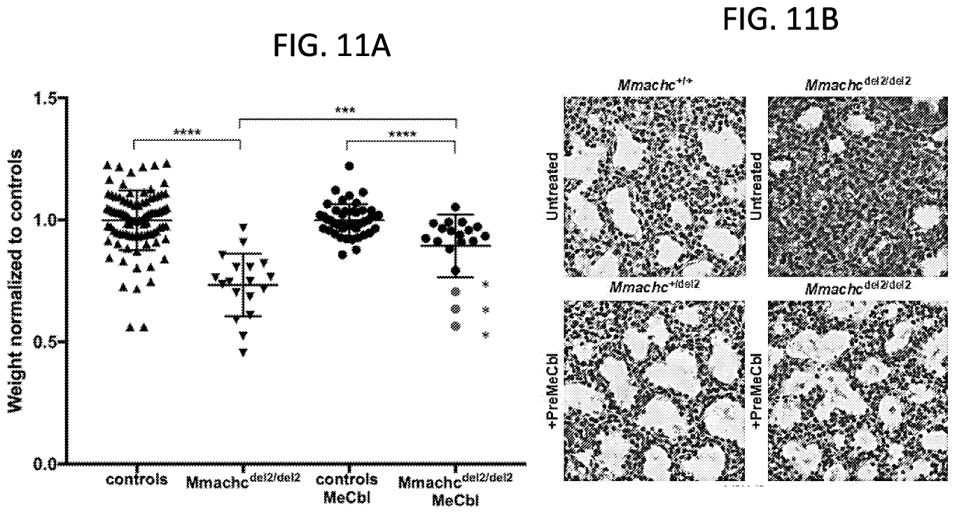

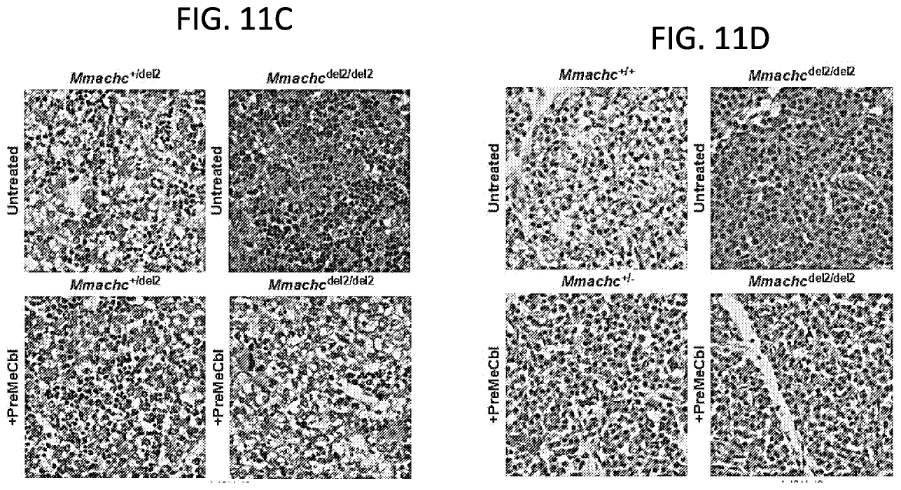

FIG. 11A shows that the Mmachc^{de12/de12} mutants born to Mmachc^{de12} dams treated with subcutaneous MeCbl injections once per week during gestation have improved weight at E18.5 compared to untreated mutants (p=0.0004). The pathology of the Mmachc^{de12/de12} mutants was also examined at E18.5 with and without prenatal MeCbl treatment (FIG. 11B-11D). The mutants had multisystemic pathology in the brain, lungs, liver and brown fat at E18.5. Prenatal MeCbl treatment ameliorated the pathology in the lungs, liver and brown fat, analysis pending in brain. This dramatic response to prenatal MeCbl in the Mmachc^{de12/de12} prompted us to compare selected metabolites with the various cobalamin treatments. The homocysteine levels did not improve with any cobalamin therapy (prenatal OHCbl, prenatal MeCbl, prenatal and weekly OHCbl) (FIG. 4C). Interestingly, it was found that prenatal MeCbl treatment reduced cystathionine to control levels (p<0.01) vs in prenatally OHCbl treated mutants, where cystathionine was elevated compared to untreated mutants (p<0.01) (FIG. 4D). Cystathionine is a metabolite of homocysteine characteristically elevated in patients with cblC due to the transsulfuration of homocysteine by cystathionine beta synthase (CBS) requiring pyroxidine (vitamin B6). B12 deficient states in rats have been shown to affect CBS activity (Uekawa et al., J Nutrigenet Nutrigenomics 2, 29-36 (2009)).

Thus, MeCbl can be superior to OHCbl in controlling the homocysteine remethylation to methionine in vivo, which may be more important for disease pathophysiology. Interestingly, MeCbl is higher than OHCbl in fetal blood in humans, suggesting a preferential or active transport across the placenta. Plasma MeCbl is also high in infancy and slowly declines through life (Craft et al., J Clin Pathol 24, 449-55 (1971); Linnell and Matthews, Clin Sci (Lond) 66, 113-21 (1984)) and therefore may be important for neonatal survival.

### Example 5

AAV Gene Therapy Treatments Improved Survival and Outcomes of Mmachc^{de13/de13} Mice

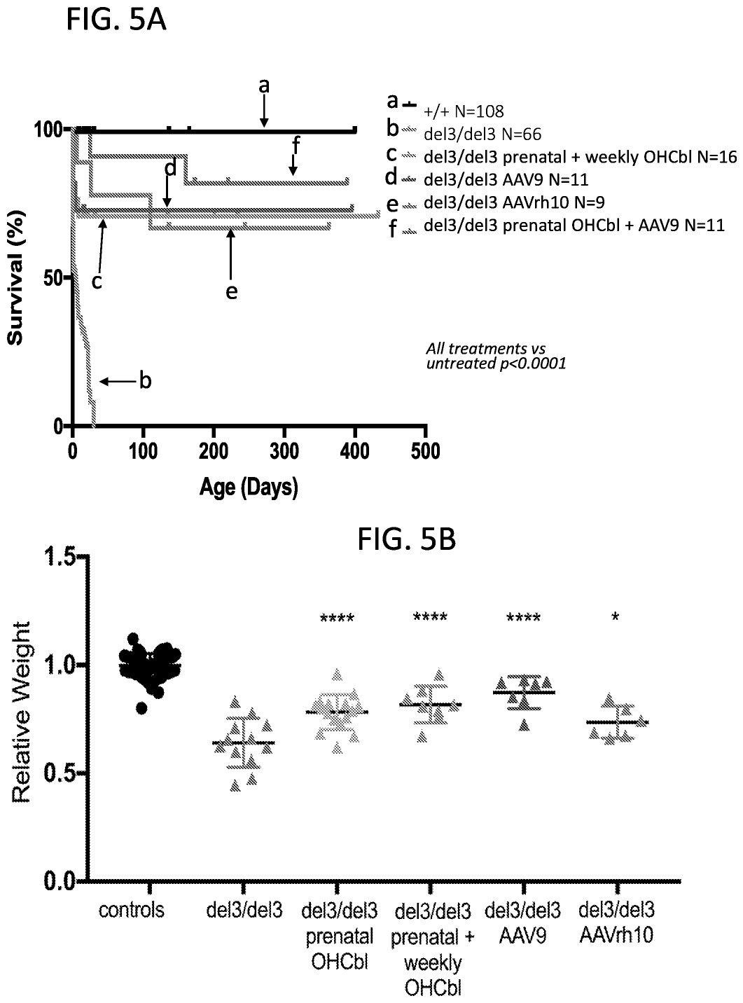

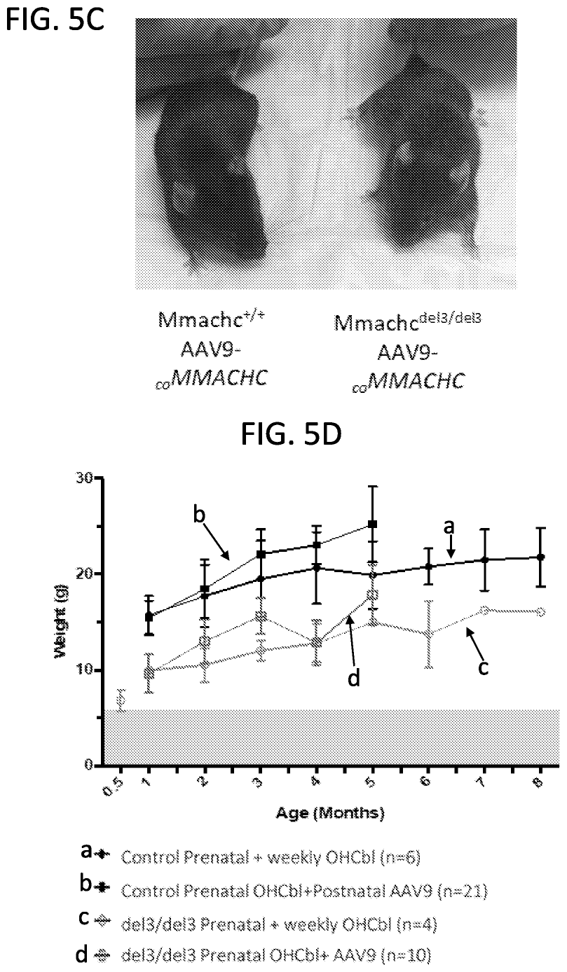

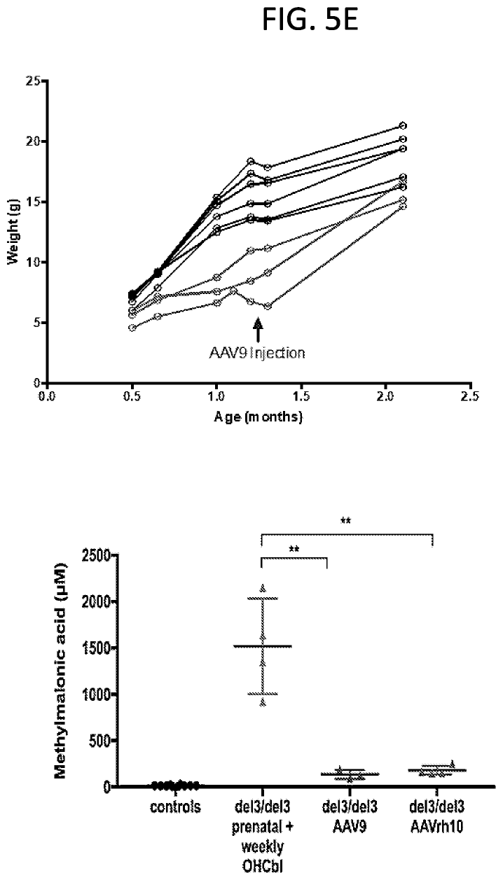

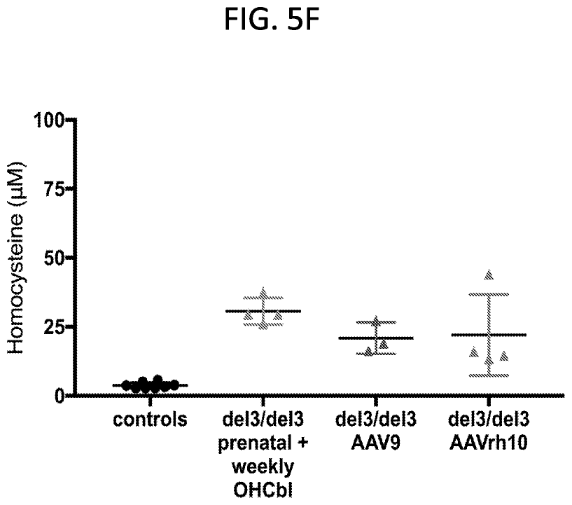

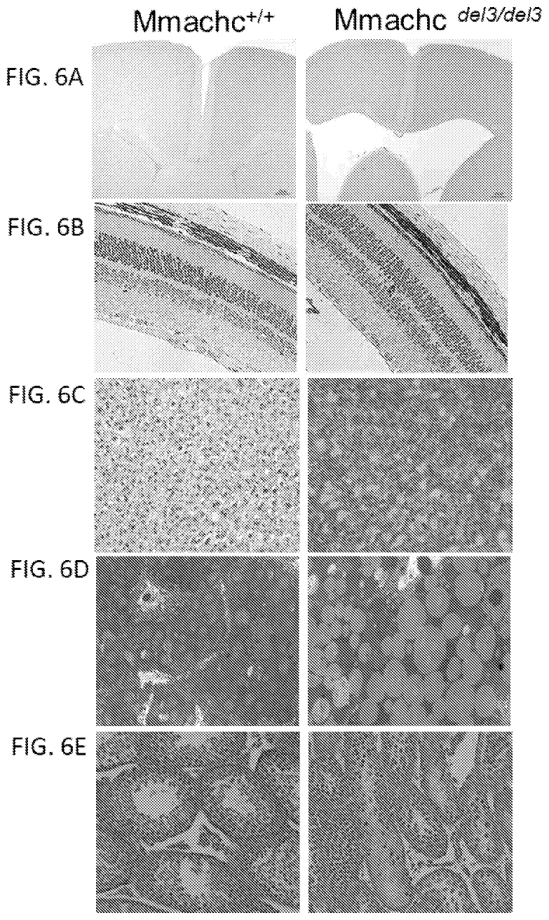

Systemic AAV gene replacement has been a successful approach to treat inborn errors of metabolism including a related mouse model of mut methylmalonic acidemia (Chandler and Venditti,. Mol Ther 18, 11-6 (2010); Chandler and Venditti, Transl Sci Rare Dis 1, 73-89 (2016)). Two AAVs were engineered: 1) a codon optimized human MMACHC with chicken β-actin promoter and AAV9 capsid 2) mouse Mmachc with chicken β-actin promoter with AAVrh10 capsid (FIG. 10). Intrahepatic injection of 1×10¹¹ genome copies/pup (˜6×10¹³/kg; average weight 1.7 g) was performed on day 0-2 of life. Similar to OHCbl therapy, both AAV treatments dramatically improved the survival of Mmachc^{de13/de13} mice (FIG. 5A) with long term survival of greater than 1 year (8/11, AAV9; 6/9, AAVrh10). Combination of prenatal OHCbl and AAV9-MMACHC showed improved neonatal survival and long-term survival of greater than 1 year (9/11, prenatal OHCbl+AAV9) although was not significantly different from AAV9 alone. Weight was improved compared with untreated mutants at two weeks, yet the treated mutants remained smaller than unaffected controls (FIG. 5B) and remain growth impaired at 5 months (FIG. 5D). A small pilot study was also performed in prenatally treated MeCbl Mmachcde12/de12 mice with retroorbital injection of AAV9 at 1 month of age which showed improvement in the weight of the mutants almost to their control littermates. Biochemical correction of the Mmachc^{de13/de13} mice was confirmed by a decrease in MMA levels at 2 weeks although MMA was still elevated compared with littermates (FIG. 10A). Long term biochemical correction of MMA was observed in adult mice treated neonatally with AAV9 (FIG. 5E) although homocysteine still remained at levels similar to untreated mice (FIG. 5F).

### Example 6

### Clinical Appearance and Pathology

Pathology of mice dying in the newborn period did not determine a specific underlying cause of death suggesting a potential metabolic etiology. Surviving untreated Mmachc^{de13/de13} (2 males, 1 female) and controls underwent full pathologic examination and laboratory analysis at 4 weeks of age. Significant histologic findings in Mmachc^{de13/de13} mice included: brain abnormalities included hypoplastic corpus callosum (3/3) and dilated lateral and third ventricles (2/3) (FIG. 5A). The adrenal gland had thinner cortices with mild disorganization of the normal cord structure in the zonal fascicularis. When mice were maintained on a high fat diet, severe hepatic lipidosis was observed by Oil Red O staining, and electron microscopy confirmed macrovesicular steatosis (FIG. 5C, D). Bones appeared to have thinner cortical and woven bone. In males, hypoplastic testes with increased numbers of apoptotic cells in seminiferous tubules was observed (FIG. 5E). Laboratory chemistry and hematologic parameters were not statistically different than controls, with the exception of glucose, which was lower in mutants but within the normal range (controls 259.3+/−46.3 vs mutants 163.7+/−51.3; p<0.04).

The severe neonatal lethality of this model in combination with the fact that the mouse retina develops until ˜P21 made it challenging to study retinal pathology in untreated mice. Retinal histology was within normal limits at 30 days in untreated mice, and at 6-9 months of age in OHCbl treated and prenatal OHCbl/AAV9 treated Mmachc^{de13/de13} mice (FIG. 4B). Electroretinography (ERG) was performed in treated Mmachc^{de13/de13} mice that showed diminished scotopic and photopic A and B waves in prenatal OHCbl/AAV9 treated mice at 9 months and diminished scotopic B waves in OHCbl treated mice at 6 months.

### Example 7

### Clinical Data

Extensive data was collected on a large natural history cohort of 61 patients studied every 2-3 years over 15 yrs to evaluate the impact of different approaches to clinical management/cobalamin therapy on disease outcomes (ClinicalTrial.gov ID: NCT00078078). The clinical data show improved neurocognitive outcomes in a small number of young children treated with higher doses of hydroxocobalamin 5-20 mg/day since infancy. Six patients (ages 2.2-3.8 years), who received higher doses of hydroxocobalamin (5-20 mg/day or 0.3-1.4 mg/kg/d) starting between 6 mo-1 yr, presented with variable macular degeneration and improved average FSIQ and ABC scores in the normal range (FSIQ 91.6±5.3; ABC 88.2±10.7), compared to our historic scores in young children <10 y of age on <0.3 mg/kg/day (FSIQ 70.69±19.11, ABC 79.83±17.42). A single patient receiving 5 mg of hydroxocobalamin since 35 days of age had a normal eye exam and an ABC score of 79 at age 3.5 years. Adults and children on 25 mg/day have been monitored closely for 2-8 years have no significant adverse events on the high dose hydroxocobalamin therapy.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A method of treating a subject with a cobalamin C (cblC) deficiency, comprising:
selecting a human subject with the cblC deficiency; and
administering about 5 mg to about 10 grams of methylcobalamin (MeCbl) daily to the human subject, thereby treating the cblC deficiency in the subject.

2. The method of claim 1, comprising administering about 10 mg to about 10 grams of MeCbl daily to the human subject.

3. The method of claim 1, wherein the human subject is an infant.

4. The method of claim 1, wherein the human subject is a child.

5. The method of claim 1, wherein the human subject is an adult.

6. The method of claim 1, wherein the human subject has macular disease or a retinal degeneration, and wherein retinal function is improved in the human subject following the administering.

7. The method of claim 1, wherein the human subject has impaired neurocognitive function, and wherein a neurocognitive outcome is improved in the human subject following the administering.

8. The method of claim 1, wherein the human subject has seizures, and wherein seizures are improved in the human subject following the administering.

9. The method of claim 1, wherein the human subject has neuropathy, and wherein neuropathy is improved in the human subject following the administering.

10. The method of claim 1, wherein the human subject has homocystinuria/hyperhomocysteinemia.

11. The method of claim 10, wherein the human subject has methylmalonic acidemia.

12. A method of treating a fetus with a cblC deficiency, comprising:
selecting a female human subject pregnant with the fetus that has the cblC deficiency; and
administering about 5 mg to about 10 grams of MeCbl daily to the female human subject, thereby treating the cblC deficiency in the fetus.

13. The method of claim 12, wherein the female human subject is in the second or third trimester.

14. The method of claim 13, wherein the fetus has intrauterine growth retardation (IUGR), and wherein growth of the fetus is improved following administration to the human subject.

15. The method of claim 12, wherein one or more of ocular function, macular function, cardiac function, kidney function, brain growth, neurocognitive function or hydrocephalus is improved in the fetus following birth as compared to a control.

16. The method of claim 15, wherein the control is the ocular function, macular function, cardiac function, kidney function, brain growth, neurocognitive function or hydrocephalus in a fetus following birth, wherein the fetus was not administered the MeCbl.

17. The method of claim 1, wherein the MeCbl is administered to the human subject at a dose of about 0.05 to about 100 mg/kg/day.

18. The method of claim 17, wherein the MeCbl is administered to the human subject at a dose of about 0.3 to about 50 mg/kg/day

19. The method of claim 1, further comprising administering to the human subject a therapeutically effective amount of hydroxocobalamin (OHCbl).

20. The method of claim 19, comprising administering about 5 mg to about 50 mg of OHCbl.

21. The method of claim 20, comprising administering to the human subject about 5 mg to about 50 mg of MeCbl and about 5 mg to about 10 grams of OHCbl as a single dose to the human subject.

22. The method of claim 1, wherein the MeCbl is administered intravenously, intramuscularly, or subcutaneously.

23. The method of claim 1, further comprising performing an assay on a biological sample from the subject, wherein the assay is
a) urine organic acid analysis
b) serum methylmalonic acid analysis;
c) total plasma homocysteine analysis;
d) plasma amino acid analysis;
e) serum vitamin B12 level;
f) plasma acylcarnitine analysis; or
g) measurement of cystathionine.

24. The method of claim 1, wherein the human subject has a cblC, epicblC, cblD-combined, cblD-homocystinuria, cblE, cblF, cblG, cblJ or cblX disorder.

25. The method of claim 1, further comprising administering to the subject an effective amount of betaine, folate and/or folinic acid.

26-39. (canceled)
